(19) United States
(12) Patent Application Publication
(10) Pub. No.: US 2004/0220273 A1
(43) Pub. Date: Nov. 4, 2004

(54) Preparation of 2-aminomethyl-5-fluorobenzamides

(76) Inventors: Jaemoon Lee, Edison, NJ (US); Yong-Li Zhong, Edison, NJ (US)

Correspondence Address: MERCK AND CO INC, P O BOX 2000, RAHWAY, NJ, 070650907

(21) Appl. No.: 10/797,496

(22) Filed: Mar. 10, 2004

Related U.S. Application Data
(60) Provisional application No. 60/454,260, filed on Mar. 12, 2003.

(51) Int. Cl. A61K 31/165; C07C 231/02

(52) U.S. Cl. 514/618; 564/134

(57) ABSTRACT

Benzamide compounds of Formula VII are prepared by reacting a benzoate compound of Formula V with an amine to obtain a benzamide compound of Formula VI, and then treating the benzamide VI with an amine deprotecting agent to obtain the benzamide VII; wherein R¹ and R² are each independently H, alkyl, substituted alkyl, cycloalkyl, substituted cycloalkyl, aryl, or substituted aryl; R³ is alkyl, -alkylaryl, or aryl; and P* is an amino protective group.

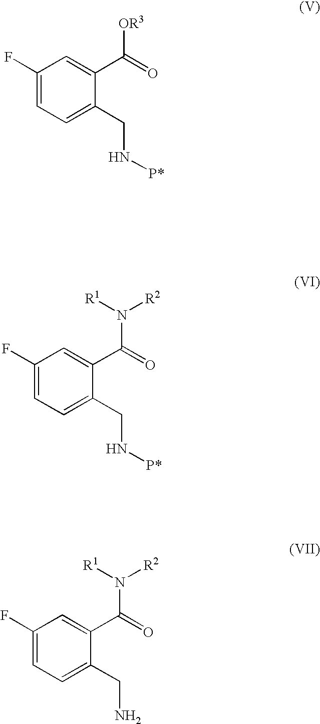

Embodiments of this preparative method include one or more of the following additional steps: obtaining the benzoate V having the N-protected ortho-aminomethyl substituent by treating the corresponding benzoate IV having a free aminomethyl substituent with an amine protecting agent, hydrogenating an ortho-cyanobenzoate III (also referred to as benzonitrile III) to obtain the benzoate IV, cyanating an ortho-halobenzoate II to obtain benzonitrile III, and esterifying an ortho-halobenzoic acid I to obtain ortho-halobenzoate II. The benzamides are Formula VII are useful as intermediates in the preparation of HIV integrase inhibitors.

This application claims the benefit of U.S. Provisional Application No. 60/454,260, filed Mar. 12, 2003, the disclosure of which is hereby incorporated by reference in its entirety.

### FIELD OF THE INVENTION

The present invention is directed to a process for preparing 2-aminomethyl-5-fluorobenzamides, which can be coupled with naphthyridine carboxylic acids or esters thereof to form naphthyridine carboxamides that are useful as HIV integrase inhibitors.

### BACKGROUND OF THE INVENTION

The HIV retrovirus is the causative agent for AIDS. The HIV-1 retrovirus primarily uses the CD4 receptor (a 58 kDa transmembrane protein) to gain entry into cells, through high-affinity interactions between the viral envelope glycoprotein (gp 120) and a specific region of the CD4 molecule found in T-lymphocytes and CD4 (+) T-helper cells (Lasky L. A. et al., Cell 1987, 50: 975-985). HIV infection is characterized by an asymptomatic period immediately following infection that is devoid of clinical manifestations in the patient. Progressive HIV-induced destruction of the immune system then leads to increased susceptibility to opportunistic infections, which eventually produces a syndrome called ARC (AIDS-related complex) characterized by symptoms such as persistent generalized lymphadenopathy, fever, and weight loss, followed itself by full blown AIDS.

After entry of the retrovirus into a cell, viral RNA is converted into DNA, which is then integrated into the host cell DNA. Integration of viral DNA is an essential step in the viral life cycle. Integration is believed to be mediated by integrase, a 32 kDa enzyme, in three steps: assembly of a stable nucleoprotein complex with viral DNA sequences; cleavage of two nucleotides from the 3′ termini of the linear proviral DNA; and covalent joining of the recessed 3′ OH termini of the proviral DNA at a staggered cut made at the host target site. The fourth step in the process, repair synthesis of the resultant gap, may be accomplished by cellular enzymes.

Certain 8-hydroxy-1,6-naphthyridine-7-carboxamides constitute a class of inhibitors of HIV integrase and of HIV replication. Compounds of this class include, but are not limited to, compounds of Formula (A):

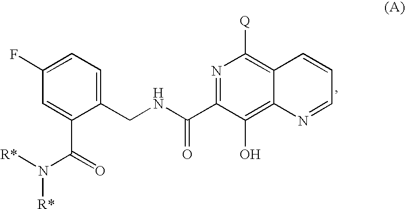

and pharmaceutically acceptable salts thereof, wherein:

each R* is independently H, alkyl, or cycloalkyl;

Q is H, —C(═O)N(R^{X}R^{Y}), —N(R^{X})SO₂R^{Z}, or 1,1-dioxido-1,2-thiazinan-2-yl;

R^{X} and R^{Y} are each independently H, alkyl, or cycloalkyl; and

R^{Z} is alkyl or cycloalkyl.

Exemplary of compounds of Formula (A) is the compound of formula:

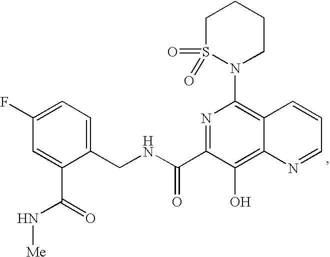

alternatively referred to herein as Compound 10.

Compounds of Formula (A) can be prepared by coupling 8-hydroxy-naphthyridine-7-carboxylic acids (or acid derivatives such as acid halides or esters) with the appropriate 2-aminocarbonyl-4-fluorobenzylamine (typically and alternatively referred to herein as the 2-aminomethyl-5-fluorobenzamide or, more simply, as the benzamide side chain). The benzamide side chain can be prepared using the method exemplified in Scheme A below.

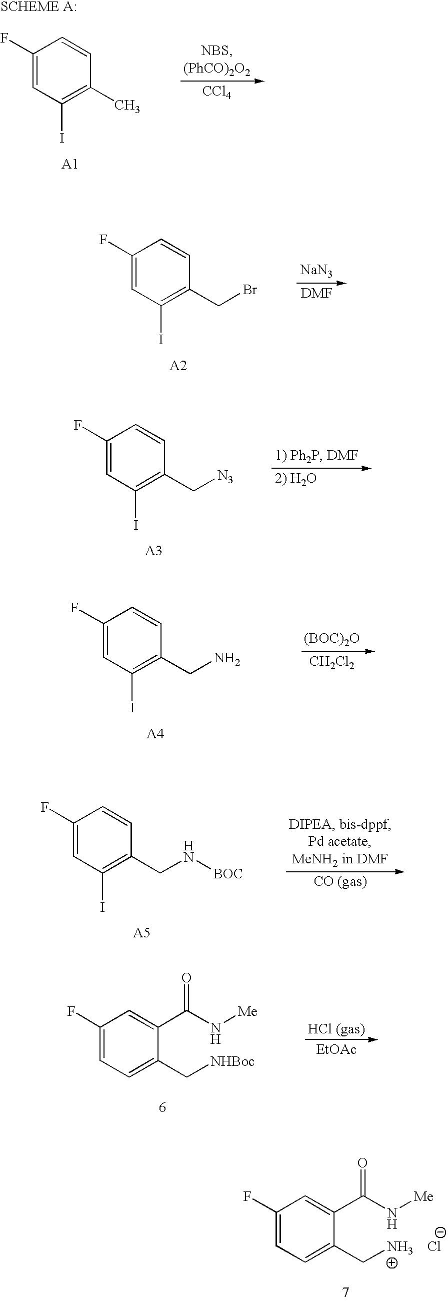

Unfortunately, the process depicted in Scheme A has several disadvantages. The starting material A1 is quite expensive and not available in bulk quantitites, and the bromination in the first step of Scheme A results in the formation of significant dibromide byproduct, requiring chromatographic purification of the product A2. The fifth step of Scheme A is an aminocarbonylation that involves the use of carbon monoxide which presents a serious safety hazard. Careful handling of the CO and monitoring of CO levels is essential. In addition, the aminocarbonylation step has a relatively low yield of 6 (e.g., about 60%), and chromatographic purification of the product is required due to the formation of significant byproduct. The overall yield observed for the process of Scheme A is typically less than 30%, which is quite low especially for the production of benzamide side chain in bulk quantities. In summary, the Scheme A process is not well suited to the large scale production of benzamide side chain.

The benzamide side chain can also be prepared using a variation of Scheme A, exemplified in Scheme B below.

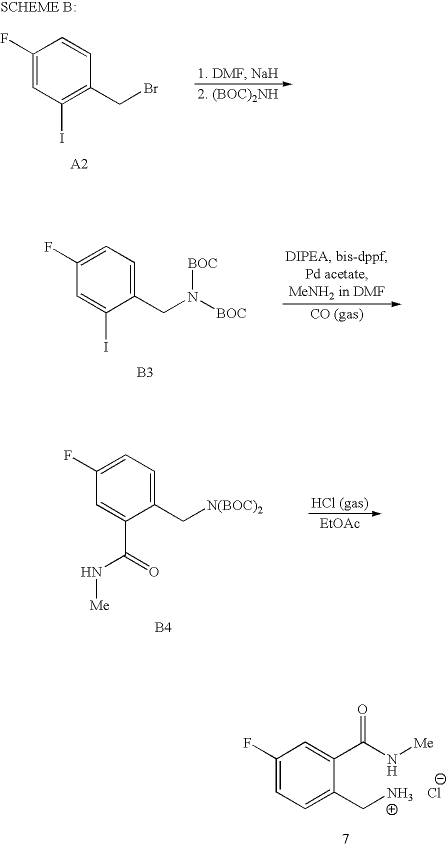

Scheme B requires fewer steps than Scheme A, but nonetheless still includes the aminocarbonylation step and its attendant disadvantages as described above. In addition, the reagent (BOC)₂NH used in the first step to prepare di-BOC intermediate B3 is very expensive and not available in bulk quantities. Overall yields for Scheme B are no better than those for Scheme A; i.e., they are typically less than 30%.

Accordingly, there is a need for more efficient methods for preparing the benzamide side chain.

### SUMMARY OF THE INVENTION

The present invention is directed to a process for preparing 2-aminomethyl-5-fluorobenzamides that can be coupled to naphthyridine carboxylic acids or esters thereof to form naphthyridine carboxamide integrase inhibitors. More particularly, the present invention is a process for preparing a benzamide compound of Formula (VII):

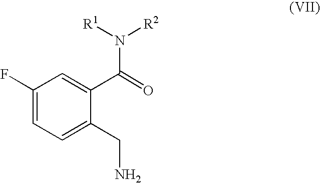

which comprises:

(Y) reacting a benzoate compound of Formula (V):

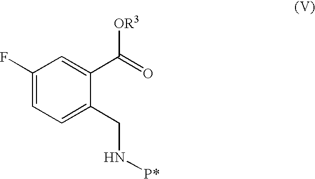

with an amine of formula R¹R²NH in a solvent Y to obtain a benzamide compound of Formula (VI):

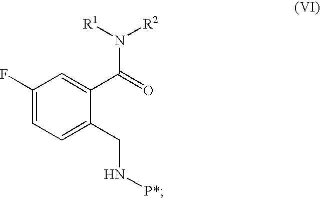

(Z) treating the benzamide compound of Formula (VI) with an amine deprotecting agent to obtain the benzamide compound of Formula (VII);

wherein:

R¹ and R² are each independently:

(1) —H,

(2) —C₁₋₆ alkyl, optionally substituted with from 1 to 5 substituents each of which is independently —OH, —O—C₁₋₆ alkyl, —CN, —NO₂, —N(R^{a})R^{b}, —C(═O)N(R^{a})R^{b}, —SO₂N(R^{a})R^{b}, —N(R^{a})C(═O)R^{b}, —N(R^{a})CO₂R^{c}, —N(R^{a})SO₂R^{c}, —N(R^{a})SO₂N(R^{a})R^{b}, —OC(═O)N(R^{a})R^{b}, or —N(R^{a})C(═O)N(R^{a})R^{b},

(3) —C₃₋₆ cycloalkyl, optionally substituted with from 1 to 4 substituents each of which is independently —C₁₋₄ alkyl or —O—C₁₋₄ alkyl, or

(4) aryl, optionally substituted with from 1 to 6 substituents each of which is independently halogen, —C₁₋₄ alkyl, —O—C₁₋₄ alkyl, —CN, —N(R^{a})R^{b}, —C(═O)N(R^{a})R^{b}, —SO₂N(R^{a})R^{b}, —N(R^{a})C(═O)R^{b}, —N(R^{a})CO₂R^{c}, —N(R^{a})SO₂R^{c}, —(CH₂)₁₋₂—O—C₁₋₄ alkyl, —(CH₂)₁₋₂—CN, —(CH₂)₁₋₂—N(R^{a})R^{b}, —(CH₂)₁₋₂—C(═O)N(R^{a})R^{b}, —(CH₂)₁₋₂—SO₂N(R^{a})R^{b}, —(CH₂)₁₋₂—N(R^{a})C(═O)R^{b}, —(CH₂)₁₋₂—N(R^{a})CO₂R^{c}, —(CH₂)₁₋₂—N(R^{a})SO₂R^{c}, phenyl, or —(CH₂)₁₋₂-phenyl;

R³ is —C₁₋₆ alkyl, —C₁₋₆ alkyl-aryl, or aryl;

P* is an amino protective group;

each R^{a} is independently —H, —C₁₋₆ alkyl, or —C₃₋₆ cycloalkyl;

each R^{b} is independently —H, —C₁₋₆ alkyl, or —C₃₋₆ cycloalkyl; and

each R^{c} is independently —C₁₋₆ alkyl or —C₃₋₆ cycloalkyl.

The process of the present invention can provide the benzamide compound of Formula (VII) in a high yield with respect to the starting benzoate compound of Formula (V). For example, the process has typically resulted in an overall yield of at least about 90% of N-methyl 2-aminomethyl-5-fluorobenzene carboxamide (alternatively referred to herein as Compound 7) from methyl 2-t-butyloxycarbonylaminomethyl-5-fluorobenzoate (alternatively referred to herein as Compound 5). The efficiency of the process of the invention is surprising, because the process would be expected to form substantial or major amounts of lactam byproduct due to cyclization of the amino group with the ester in Compound V and with the amide in Compound VI.

An embodiment of the invention is the process of the invention as set forth above, further comprising Steps U, V, W and X as described below, wherein compounds of Formula (V) are prepared starting from 5-fluoro-2-halobenzoic acids. The 5-fluoro-2-halobenzoic acids are either available commercially at relatively low cost or are typically easy to prepare in good yields or both. This multi-step process (i.e., Steps U, V, W, and X as described below plus Steps Y and Z as set forth above and more fully described below) can achieve yields of Compound VII of greater than 60%, a substantial improvement over the processes depicted in Schemes A and B above. In addition, this multi-step process does not include a carbonylation step and thus avoids the use of carbon monoxide, the use of which is a significant drawback to the processes of Schemes A and B.

Other embodiments, aspects and features of the present invention are either further described in or will be apparent from the ensuing description, examples and appended claims.

### DETAILED DESCRIPTION OF THE INVENTION

A benzamide compound of Formula (VII) is alternatively referred to herein more simply as “Compound VII” or “benzamide VII”. Similarly, a benzamide compound of Formula (VI) is alternatively referred to as “Compound VI” or “benzamide VI”, and a benzoate compound of Formula (V) is alternatively referred to as “Compound V” or “benzoate V”. Analogous nomenclature is employed for compounds of Formula (I) to (IV) set forth in the description below.

The present invention is directed to processes for preparing 2-aminomethyl-5-fluorobenzamides, which are useful as the side chains of naphthyridine carboxamide integrase inhibitors. The present invention includes the process comprising Steps Y and Z as set forth above in the Summary of the Invention.

An embodiment of the present invention is the process comprising Steps Y and Z as set forth above, wherein R¹ and R² in the definition of Compounds VI and VII are each independently —H, —C₁₋₆ alkyl, —C₃₋₆ cycloalkyl, or aryl. In an aspect of this embodiment, R¹ and R² are each independently —H, —C₁₋₄ alkyl, cyclopropyl, or phenyl. In another embodiment of the present invention, R¹ and R² are each independently —H or —C₁₋₆ alkyl. In an aspect of this embodiment, R¹ and R² are each independently —H or —C₁₋₃ alkyl. In still another embodiment, one of R¹ and R² is —H and the other of R¹ and R² is —C₁₋₆ alkyl. Other embodiments include the process comprising Steps Y and Z in which R¹ is —H and R² is —C₁₋₄ alkyl; or R1 is H and R2 is methyl, ethyl, n-propyl, or isopropyl; or R1 is H and R2 is methyl or ethyl; or R1 is H and R2 is methyl.

Another embodiment of the present invention is the process comprising Steps Y and Z as set forth above, wherein R³ is —C₁₋₆ alkyl, —CH₂-aryl, or aryl. In another embodiment, R³ is —C₁₋₄ alkyl, benzyl, or phenyl. Other embodiments include the process comprising Steps Y and Z in which R3 is —C₁₋₄ alkyl; or is methyl, ethyl, n-propyl, or isopropyl; or is methyl or ethyl; or is methyl.

Another embodiment of the present invention is the process comprising Steps Y and Z as set forth above, wherein P* is

(1) —C(═O)—O—C₁₋₆ alkyl,

(2) —C(═O)—O—CH₂-aryl,

(3) —C(═O)—O—(CH₂)₀₋₁—CH═CH₂,

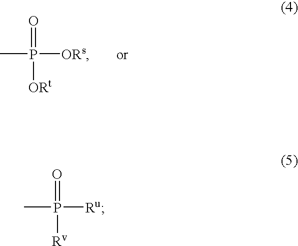

wherein R^{s} and R^{t} are each independently —C₁₋₆ alkyl, —CH₂-aryl, or aryl ; and

R^{u} and R^{v} are each independently an aryl group.

Aspects of this embodiment include P* as defined above, wherein R^{s} and R^{t} are each independently —C₁₋₄ alkyl, benzyl, or phenyl; or R^{s} is the same as R^{t} (i.e., R^{s} and R^{t} are both the same —C₁₋₆ alkyl group, the same —CH₂-aryl, or the same aryl); or R^{s} and R^{t} are both phenyl, or both benzyl, or both the same —C₁₋₄ alkyl group (e.g., both methyl, both ethyl, both n-propyl, both isopropyl, both n-butyl, or both t-butyl). Other aspects of this embodiment include P* as defined above, wherein R^{u} and R^{v} are both the same aryl group; or R^{u} and R^{v} are both phenyl.

Another embodiment of the present invention is the process comprising Steps Y and Z, wherein P* is selected from the group consisting of (C₁₋₄ alkyloxy)carbonyl, benzyloxycarbonyl (CBZ), allyloxycarbonyl (ALLOC), diphenylphosphinyl, di—(C₁₋₃ alkyl)phosphono, diphenylphosphono, and dibenzylphosphono. In another embodiment, P* is t-butyloxycarboxnyl (BOC), CBZ, or ALLOC. In still another embodiment, P* is BOC.

Certain of the substituents set forth in the definitions of R¹ and R² herein include groups R^{a} and R^{b}. Each R^{a} and R^{b} is independently —H, —C₁₋₆ alkyl, or —C₃₋₆ cycloalkyl. In one embodiment, each R^{a} and R^{b} is independently —H or —C₁₋₄ alkyl. In another embodiment, each R^{a} and R^{b} is independently —H or —C₁₋₃ alkyl. In another embodiment, each R^{a} and R^{b} is independently —H, methyl, or ethyl. In still another embodiment, each R^{a} and R^{b} is independently —H or methyl.

Certain of the substituents set forth in the definitions of R¹ and R² include the group R^{c}. Each R^{c} is independently a —C₁₋₆ alkyl or a —C₃₋₆ cycloalkyl. In one embodiment, each R^{c} is independently a —C₁₋₄ alkyl. In another embodiment, each R^{c} is independently a —C₁₋₃ alkyl. In another embodiment, each R^{c} is independently methyl or ethyl. In still another embodiment, each R^{c} is methyl.

It is understood that any embodiment, aspect, or feature of any one of P*, R¹, R², R³, R^{a}, R^{b}, and R^{c} can be combined with any embodiment, aspect of feature of any one or more of the others of P*, R¹, R², R³, R^{a}, R^{b}, and R^{c}. Each such possible combination, when incorporated into the process of the invention as defined above, represents an embodiment of the process of the present invention.

In Step Y an amine of formula R¹R²NH is reacted (i.e., acylated) with benzoate compound V in a solvent Y to obtain benzamide compound VI. The solvent Y can suitably be selected from the group consisting of aromatic hydrocarbons, halogenated aliphatic hydrocarbons, alcohols, ethers, and nitriles. In one embodiment, the solvent Y is selected from the group consisting of C₆-C₁₄ aromatic hydrocarbons, dialkyl ethers wherein each alkyl is independently a C₁-C₆ alkyl, C₁-C₆ linear and branched alkanes substituted with two —O—C₁-C₆ alkyl groups (which are the same or different), C₄-C₈ cyclic ethers and diethers, C₆-C₈ aromatic ethers, and C₂-C₆ aliphatic nitriles. Exemplary solvents for use in Step Y include benzene, toluene, o-, m-, and p-xylene (single or mixed isomers), ethylbenzene, carbon tetrachloride, chloroform, methylene chloride, 1,2-dichloroethane, 1,1,2-trichloroethane, 1,1,2,2-tetrachloroethane, methanol, ethanol, propanol, isopropanol, n-butanol, isobutanol, THF, DME, MTBE, di-n-butyl ether, dioxane, acetonitrile, and propionitrile.

In another embodiment, the solvent Y is selected from aromatic hydrocarbons, alcohols, and ethers. In an aspect of the preceding embodiment, the solvent Y is selected from the group consisting of C₁-C₆ alkyl alcohols, dialkyl ethers wherein each alkyl is independently a C₁-C₄ alkyl, C₄-C₅ cyclic ethers, and C₇-C₈ aromatic hydrocarbons. In another aspect of the preceding embodiment, the solvent Y is methanol, ethanol, n-propanol, isopropanol, n-butanol, diethylether, THF, DME, toluene, or single or mixed isomers of xylene. In still another aspect of the preceding embodiment, solvent Y is toluene or single or mixed isomers of xylene.

The amine of formula R¹R²NH can be employed in Step Y in any proportion with respect to Compound V which will result in the formation of at least some of Compound V, but is typically employed in an amount that can optimize conversion of Compound V and formation of Compound VI. In one embodiment, the amine is employed in Step Y in an amount in a range of from about 1 to about 200 equivalents per equivalent of benzoate V. In another embodiment, the amine is employed in an amount in a range of from about 1 to about 50 (e.g., from about 1 to about 10) equivalents per equivalent of Compound V. In still another embodiment, the amine is employed in an amount in a range of from about 1 to about 5 (e.g., from about 1.5 to 5) equivalents per equivalent of Compound V. In still another embodiment, the amine is employed in an amount in a range of from about 2 to about 5 equivalents per equivalent of Compound V.

Step Y can be conducted at any temperature at which the reaction (acylation) to form Compound VI can be detected. The temperature is suitably in a range of from about 50 to about 200° C., and the reaction is typically conducted at a temperature in a range of from about 75 to about 150° C. (e.g., from about 75 to about 125° C.). In one embodiment, the temperature is in a range of from about 75 to about 100° C.

The Step Y reaction can be conducted by charging the solvent Y, the amine, and Compound V to a suitable reaction vessel, bringing the resulting mixture to reaction temperature, and maintaining the mixture at reaction temperature (optionally with agitation such as stirring) until the reaction is complete or the desired degree of conversion of the reactants is achieved. The order of addition of the reactants and reagents to the reaction vessel is typically not critical; i.e., they can be charged concurrently or sequentially in any order. For example, Compound V can first be dissolved in solvent Y, and the solution charged to the reaction vessel, followed by addition of the amine. When the amine is a gas (e.g., methylamine), the reaction can be conducted under pressure in a suitable reactor (e.g., a bomb). The reaction time can vary widely depending upon, inter alia, the reaction temperature and the choice and relative amounts of reactants, but the reaction time is typically in a range of from about 1 to about 96 hours. Compound VI can subsequently be isolated (alternatively referred to as recovered) from the reaction mixture using conventional procedures, such as by cooling and concentrating the post-reaction mixture to precipitate the desired product, then separating the product by filtration.

In Step Z, the benzamide compound of Formula (VI) is treated with an amine deprotecting agent to obtain the benzamide compound of Formula (VII). The amino protective group P* in Compounds V and VI can be any amino protective group that is stable enough to survive the acylation of Step Y and labile enough to be removed (cleaved) from Compound VI via contact with a suitable amine deprotecting agent to form benzamide VII with little or no degradation of the amido group (e.g., little or no lactam formation). Suitable P* groups include alkyloxycarbonyls (e.g., BOC), arylmethyloxycarbonyls (e.g., CBZ), vinyloxycarbonyl, ALLOC, diarylphosphinyls, diarylphosphonos, and dialkylphosphonos, such as those defined and described earlier. These P* groups can be formed by treating the amine precursors of Compound V with amine protecting agents. Suitable amine protecting agents and treatment methods are described below in the discussion of Step X. In most instances the P* groups can be removed by treatment with acids including mineral acids, Lewis acids, and organic acids. Suitable mineral acids include hydrogen halides (HCl, HBr, and HF, as a gas or in aqueous solution), sulfuric acid, and nitric acid. Suitable organic acids include carboxylic acids, alkylsulfonic acids and arylsulfonic acids. Exemplary organic acids include trifluoroacetic acid (TFA), toluenesulfonic acid, benzenesulfonic acid, methanesulfonic acid, and trifluoromethanesulfonic acid. Suitable Lewis acids include BF₃.Et₂O, SnCl₄, ZnBr₂, Me₃SiI, Me₃SiCl, Me₃SiOTf, and AlCl₃. Cleavage conditions (e.g., temperature, choice and concentration of acid) can vary from mild to harsh depending upon the lability of the amino protective group. Although acid treatment is typically effective, other means can often be employed. Removal of CBZ or ALLOC, for example, is typically accomplished via hydrogenolysis (e.g., hydrogenation with a Pd catalyst). Further description of amine deprotecting agents and deprotection treatments suitable for use in Step Z can be found in Protective Groups in Organic Chemistry, edited by J. F. W. McOmie, Plenum Press, New York, 1973, pp. 43-74; and in T. W. Greene and P. G. M. Wuts, Protective Groups in Organic Synthesis, 2^{nd} edition, John Wiley, New York, 1991, pp. 309-385.

An embodiment of the present invention is the process comprising Steps Y and Z as originally described above or as described in any of the preceding embodiments thereof, wherein P* is an amino protective group capable of being cleaved by an acid and the amine deprotecting agent in Step Z comprises an acid Z. In an aspect of this embodiment, the acid Z is a protonic acid (i.e., a proton-donating substance, also referred to in the art as a Lowry-Bronsted acid). In a feature of this aspect, the protonic acid is a mineral acid (e.g., HCl).

The treatment in Step Z (e.g., hydrogenolysis, acid hydrolysis, etc.) can be conducted at any temperature at which the formation of Compound VI can be detected. The temperature is suitably in a range of from about −50 to about 150° C., and is typically in a range of from about −50 to about 100° C. When the deprotecting agent is an acid Z, the treatment in Step Z is more typically conducted at a temperature in a range of from about −20 to about 50° C. (e.g., from about −10 to about 30° C.). When the deprotecting agent is hydrogen (for hydrogenolysis), the treatment temperature is more typically in a range of from about 0 to about 50° C. (e.g., from about 5 to about 30° C.).

When an acid Z is employed as the deprotecting agent in Step Z, it is suitably employed in an amount in a range of from about 0.1 to about 100 (e.g., from about 1 to about 50) equivalents per equivalent of benzamide VI, and is typically employed in an amount in a range of from about 0.5 to about 50 equivalents (e.g., from about 1 to 10) equivalents per equivalent of benzamide VI. In one embodiment, the acid is employed in an amount in a range of from about 1 to about 15 (e.g., from about 3 to about 15) equivalents per equivalent of benzamide VI. For hydrogenolyses, hydrogen is typically employed in an amount of at least about 1 equivalent per equivalent of benzamide VI.

The treatment in Step Z is typically conducted in solvent, hereinafter alternatively referred to as solvent Z. When treatment is with an acid, suitable solvents include esters, alcohols, halogenated aliphatic hydrocarbons, ethers, and nitriles. Suitable and exemplary alcohols, halogenated aliphatic hydrocarbons, ethers, and nitriles for Step Z are the same as those described above for Step Y. Suitable esters include C₁-C₆ alkyl esters of C₁-C₆ alkylcarboxylic acids. In one embodiment, solvent Z is a C₁₋₄ alkyl acetate (e.g., ethyl acetate, isopropyl acetate, n-butyl acetate, or isobutyl acetate). When hydrogenolysis is employed, suitable solvents include the C₁-C₆ alkyl alcohols, such as methanol, ethanol, n-propanol, and isopropanol.

The Step Z reaction can be conducted by first charging a mixture of solvent and Compound VI to a suitable reaction vessel at low temperature, then adding the amine deprotecting agent (e.g., acid Z, either as a gas such as gaseous HCl or in aqueous solution), warming the mixture to reaction temperature, and maintaining the mixture at reaction temperature (optionally with agitation) until the reaction is complete or the desired degree of conversion is achieved. When hydrogenolysis is employed, the treatment is typically conducted in a pressurized reactor. Treatment times can vary widely depending upon, inter alia, the reaction temperature and the choice and relative amounts of amine deprotecting agent and Compound VI, but the reaction time is typically in a range of from about 0.5 to about 12 hours. Compound VII can be recovered using conventional means in the form of an acid salt (e.g., a hydrochloride salt) or as the free base. Either the salt or the free base can be employed in the preparation of naphthyridine carboxamide integrase inhibitors. The acid salt is typically more stable than the free base, and thus, if the product is to be stored before use, it is usually preferred to isolate the compound as a salt.

The present invention includes a process for preparing a benzamide compound of Formula (VII) which comprises Steps Y and Z as described above and which further comprises:

(X) treating a benzoate compound of Formula (IV):

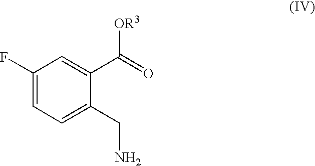

with an amine protecting agent containing the group P* in a solvent X to obtain the benzoate compound of Formula (V).

Suitable amine protecting agents for use in Step X include:

(i) compounds of formula P^{a*}-Q, wherein Q is halide (e.g., chloride or bromide) and P^{a*} is selected from the group consisting of:

(1) —C(═O)—O—C₁₋₆ alkyl,

(2) —C(═O)—O—CH₂-aryl,

(3) —C(═O)—O—(CH₂)₀₋₁—CH═CH₂,

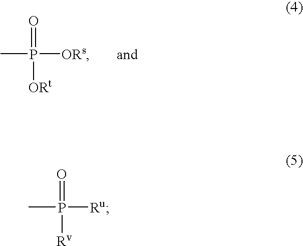

wherein R^{s} and R^{t} are each independently —C₁₋₆ alkyl, —CH₂-aryl, or aryl; and

R^{u} and R^{v} are each independently an aryl group; and

(ii) anhydrides of formula (P^{b*})₂O, wherein P^{b*} is BOC, CBz, or ALLOC.

P^{a*} and P^{b*} represent sub-definitions of P*.

A class of suitable amine protecting agents is selected from (i) compounds of formula P^{a*}-Q, wherein P^{a*} is selected from the group consisting of (C₁₋₄ alkyloxy)carbonyl, benzyloxycarbonyl (CBZ), allyloxycarbonyl (ALLOC), diphenylphosphinyl, di—(C₁₋₃ alkyl)phosphono, diphenylphosphono, and dibenzylphosphono and (ii) compounds of formula (P^{b*})₂O, wherein P^{b*} is BOC, CBZ, or ALLOC. Representative examples of amine protecting agents in this class are Ph₂P(═O)Cl, (i-PrO)₂P(═O)Cl, (t-BuO)₂P(═O)Cl, (BnO)₂P(═O)Cl, BOC-Cl, CBZ-Cl, (CBZ)₂O, (ALLOC)₂O, allyl chloroformate, and (BOC)₂O. A sub-class of this class consists of amine protecting agents selected from BOC-Q and (BOC)₂O.

Each of the aspects restricting the values of R^{s} and R^{t} and of R^{u} and R^{v} in the definition of P* as set forth in the above discussion of Steps Y and Z represent additional classes of suitable amine protecting agents of formula P^{a*}-Q.

Treating Compound IV with a compound of formula P^{a*}-Q will result in the acylation, phosphonylation, or phosphinylation of the amino group to give the corresponding carbamate (i.e., —NH—P^{a*} wherein P^{a*} is one of groups (i)(1), (i)(2) or (i)(3)), phosphoramidate (—NH—P^{a*} wherein P^{a*} is group (i)(4)), or phosphinamide (—NH—P^{a*} wherein P^{a*} is group (i)(5)). Treatment with the anhydride (P^{b*})₂O results in the acylation of the amine group to form the carbamate —NH—P^{b*}. Further description of these and other amine protecting agents suitable for use in Step X can be found in Protective Groups in Organic Chemistry, edited by J. F. W. McOmie, Plenum Press, New York, 1973, pp. 43-74; and in T. W. Greene and P. G. M. Wuts, Protective Groups in Organic Synthesis, 2^{nd} edition, John Wiley, New York, 1991, pp. 309-385; the disclosures of which are hereby incorporated by reference in their entireties.

The amine protecting agent is typically employed in an amount that can optimize conversion of benzoate compound IV to benzoate compound V. The amine protecting agent is suitably employed in an amount in a range of from about 0.9 to about 10 equivalents per equivalent of benzoate compound IV, and is typically empoyed in an amount in a range of from about 0.9 to about 3 (e.g., from about 1.1 to about 3) equivalents per equivalent of benzoate compound IV.

The treatment in Step X can be conducted at any temperature at which the reaction to form Compound V can be detected. The temperature is suitably in a range of from about −20 to about 60° C., and is typically in a range of from about −20 to about 50° C. (e.g., from about −5 to about 35° C.).

Step X is conducted in solvent X. Suitable solvents include aromatic hydrocarbons, halogenated aliphatic hydrocarbons, alcohols, esters, ethers, and nitriles. Further description of these solvent classes is set forth above in the discussion of Steps Y and Z, is applicable here, and is incorporated herein by reference. Aliphatic hydrocarbons (e.g., C₃-C₁₂ linear and branched alkanes) and alicyclic hydrocarbons (e.g., C₅-C₇ cycloalkanes), not heretofore described, are also suitable for employment as solvent X. Exemplary solvents include hexane (pure and mixed isomers), cyclohexane, cycloheptane, toluene, single and mixed isomers of xylene, methylene chloride, DCE, chloroform, carbon tetrachloride, methanol, ethanol, isopropanol, n-butanol, t-butanol and iso-butanol, ethyl acetate, isopropyl acetate, isobutyl acetate, n-butyl acetate, THF, diethyl ether, di-n-butyl ether, MTBE, DME, acetonitrile, and propionitrile.

An embodiment of the present invention is the process comprising Steps X, Y and Z, wherein the amine protecting agent in Step X is P^{a*}-Q or (P^{b*})₂O as originally defined above or as defined in a class or sub-class thereof; the amine protecting agent is employed in an amount in a range of from about 0.9 to about 10 equivalents per equivalent of benzoate compound compound IV; the solvent X is selected from the group consisting of aromatic hydrocarbons, halogenated aliphatic hydrocarbons, alcohols, ethers, and acetates; and the treatment in Step X is conducted at a temperature in a range of from about −20 to about 60° C. In an aspect of this embodiment, the amine protecting agent is BOC-Q or (BOC)₂O.

The Step X treatment can be conducted by charging the solvent X, the amine protecting agent, and Compound IV to a suitable reaction vessel, bringing the resulting mixture to reaction temperature, and maintaining the mixture at reaction temperature (optionally with agitation such as stirring) until the reaction is complete or the desired degree of conversion of the reactants is achieved. Compound IV can be charged to the vessel in the form of an acid salt or free base. When charged as an acid salt, sufficient base is typically included in the reaction mixture to neutralize the salt. Suitable bases include tertiary alkyl amines (e.g., NMM and TEA), alkali metal carbonates (e.g., sodium carbonate and potassium carbonate), and alkali metal bicarbonates (e.g., sodium bicarbonate and potassium bicarbonate). The order of addition of the reactants and reagents to the reaction vessel is typically not critical; i.e., they can be charged concurrently or sequentially in any order. For example, Compound IV can first be dissolved in solvent X, and the solution charged to the reaction vessel, followed by addition of the amine protecting agent and, when Compound IV is employed as an acid salt, the base. The reaction time can vary widely depending upon, inter alia, the reaction temperature and the choice and relative amounts of reactants and reagents, but the reaction time is typically in a range of from about 1 to about 48 hours. Compound V can subsequently be recovered from the reaction mixture by conventional means. Alternatively, the reaction mixture of Compound V in solvent X, after suitable washing and other treatment to remove impurities and unreacted reagent, can be employed directly in Step Y.

The present invention includes a process for preparing a benzamide compound of Formula (VII) which comprises Steps X, Y and Z as described above and which further comprises:

(W) hydrogenating a benzonitrile of Formula (III):

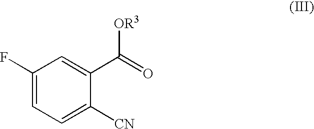

in a solvent W and in the presence of a transition metal catalyst to obtain the benzoate compound of Formula (IV).

Solvent W can suitably be selected from the group consisting of alcohols, ethers, and esters. Further description of these solvent classes is set forth above in the discussion of other process steps, is applicable here, and is incorporated herein by reference. In one embodiment, solvent W is an alcohol. In an aspect of this embodiment, solvent W is a C₁-C₆ alkyl alcohol. In another aspect of this embodiment, solvent W is a C₁-C₄ alkyl alcohol (e.g., methanol, ethanol, n-propanol, isopropanol, or isobutanol).

The hydrogenation of the benzonitrile III can be conducted over a wide range of temperatures, although the temperature is typically in the range of from about 0 to about 100° C. (e.g., from about 10 to about 100° C.). In one embodiment, the temperature is in the range of from about 15 to about 60° C. In another embodiment, the temperature is from about 25 to about 45° C.

The pressure is not a critical aspect in Step W, although atmospheric and superatmospheric pressures tend to be expedient. In one embodiment, the pressure is at least about 2 psig (115 kPa). In another embodiment, the pressure is in the range of from about 10 psig (170 kPa) to about 1,000 psig (6996 kPa).

The hydrogenation catalyst employed in Step W comprises a transition metal or a compound thereof, and is suitably a transition metal of Group VIII of the periodic table of the elements or a compound thereof. A class of suitable hydrogenation catalysts consists of catalysts selected from Pd, Ni, Pt, Rh, Ru and compounds thereof. A sub-class of suitable hydrogenation catalysts consists of catalysts selected from Pd, Pt, and compounds thereof. Exemplary of the catalysts in this sub-class are Pd, Pt, Pt halides (e.g., PtCl₂), Pd acetate, PdO, and PtO. The catalysts can be supported or unsupported. Another sub-class of suitable catalysts consists of supported and unsupported palladium catalysts. Suitable catalyst supports include carbon, silica, alumina, silicon carbide, aluminum fluoride, and calcium fluoride. Exemplary palladium catalysts include Pd black (i.e., fine metallic palladium particles) and Pd/C (i.e., palladium on a carbon support). Pd/C is a preferred catalyst.

Another sub-class of suitable hydrogenation catalysts consists of nickel catalysts. Exemplary of the catalysts in this sub-class are Raney nickel and nickel boride. Raney nickel is a preferred catalyst.

The hydrogen source is typically hydrogen gas, optionally in admixture with a carrier gas that is inert under the conditions employed in Step W (e.g., nitrogen or a noble gas such as helium or argon).

The hydrogenation in Step W is typically conducted under acidic conditions in the presence of a protonic acid W, except when the catalyst is Raney nickel. Higher yields of Compound IV have been achieved in Step W when the hydrogenation is conducted (e.g., with a Pd catalyst) in the presence of a protonic acid relative to yields under neutral or basic conditions. While not wishing to be bound by any particular chemical theory or mechanism, it is believed that the presence of a protonic acid results in the protonation of the product amine IV, which prevents it from condensing with partially reduced imine to form a secondary amine side product. In addition, rapid cyclization of the unprotonated amine product IV is avoided by the use of protonic acid during Step W. Protonic acids suitable for use in Step W include mineral acids and organic acids, such as those described earlier in the discussion of amine deprotecting agents employed in Step Z. Particularly suitable protonic acids are the hydrogen halides, especially HCl.

When the catalyst is Raney nickel, the hydrogenation is typically conducted under neutral or basic conditions.

The hydrogenation can be carried out in a pressurized reactor (e.g., an autoclave equipped with a stirrer or rocker to agitate the mixture) in which the mixture of gas (i.e., hydrogen optionally mixed with an inert gas), solvent W, benzonitrile III, catalyst, and (optionally) protonic acid W is continuously agitated. The order of addition of benzonitrile III, solvent, acid, and hydrogenation catalyst to the reaction vessel is not critical. The reactants and reagents can, for example, be added concurrently, either together or separately, or they can be added sequentially in any order. In one embodiment, benzonitrile III pre-mixed with the solvent is charged to the reaction vessel followed by addition of acid and then the catalyst. The hydrogenation can then be conducted by charging hydrogen gas, optionally in admixture with one or more inert gases, to the vessel, and then agitating the mixture under reaction conditions. The reaction time can vary widely depending upon, inter alia, the reaction temperature and pressure, the choice and relative amounts of catalyst and benzonitrile reactant, but the reaction time is typically in a range of from about 1 to about 72 hours.

Any amount of catalyst, hydrogen and protonic acid W can be employed that results in the formation of at least some of benzoate compound IV. Of course, the maximum conversion of Compound III and maximum yield of Compound IV is normally desired, and relative proportions of reactants and reagents suitable for this purpose are typically employed.

The catalyst is suitably employed in Step W in an amount in a range of from about 0.001 to about 1 equivalent per equivalent of benzonitrile III, and is typically employed in an amount in a range of from about 0.01 to about 0.8 equivalent per equivalent of benzonitrile III. In one embodiment, the catalyst (e.g., Pd- or Pt-containing catalyst) is employed in an amount in a range of from about 0.02 to about 0.5 (e.g., from about 0.02 to about 0.2) equivalents per equivalent of benzonitrile III. In another embodiment, the catalyst is employed in an amount in a range of from about 0.02 to about 0.1 (e.g., from about 0.02 to about 0.08) equivalents per equivalent of benzonitrile III.

The uptake of hydrogen is not a critical process parameter, although at least a stoichiometric amount of hydrogen gas is typically employed.

When used in Step W, the protonic acid is suitably employed in an amount of at least about 1 equivalent per equivalent of benzonitrile III, and is typically employed in an amount in a range of from about 1.1 to about 5 (e.g., from about 1.1 to about 3) equivalents per equivalent of benzonitrile III. In one embodiment, the protonic acid is employed in an amount in a range of from about 1.5 to about 3 (e.g., from about 1.5 to about 2.5) equivalents per equivalent of benzonitrile III.

The present invention includes a process for preparing a benzamide compound of Formula (VII) which comprises Steps W, X, Y and Z as described above and which further comprises:

(V) reacting a halobenzoate compound of Formula (II):

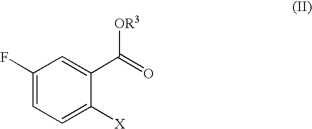

in an aprotic solvent V with a cyanide compound selected from the group consisting of CuCN and Zn(CN)₂ to obtain the benzonitrile of Formula (III); with the proviso that when the cyanide compound is Zn(CN)₂, the reaction is conducted in the presence of a Pd compound and an activating ligand; wherein X is chloro, bromo, or iodo. In one embodiment of Step V, X is Br or Cl. In an aspect of this embodiment, X is Br.

CuCN can be employed per se in the reaction, but Zn(CN)₂ is employed in the presence of a Pd compound such as with Pd₂(dba)₃ or Pd(PPh₃)₄, and an activating ligand such as dppf, PPh₃, dppe, dppp, dppb, and BINAP. Although not required, the reaction with CuCN can also be conducted in the presence of a Pd compound and an activating ligand.

Any amount of cyanide compound can be employed that results in the formation of at least some of benzonitrile compound III, but of course a high conversion of Compound II and a maximum yield of Compound III is normally desired, and a relative proportion of the cyanide compound to halobenzoate II suitable for this purpose is typically employed. The cyanide compound is suitably employed in an amount in a range of from about 0.5 to about 30 equivalents per equivalent of halobenzoate II, and is typically employed in an amount in a range of from about 0.5 to about 10 (e.g., from about 0.5 to about 5) equivalents per equivalent of halobenzoate II. In one embodiment, the cyanide compound is employed in an amount in a range of from about 0.9 to about 2 (e.g., from about 0.9 to about 1.5) equivalents per equivalent of halobenzoate II.

When used, the Pd compound is suitably employed in an amount in a range of from about 0.00001 to about 0.2 equivalents per equivalent of halobenzoate II, and is typically employed in an amount in a range of from about 0.0005 to about 0.05 equivalents per equivalent of the cyanide compound. When used, a ligand is suitably employed in an amount in a range of from about 0.001 to about 0.2 equivalents per equivalent of halobenzoate II, and is typically employed in an amount in a range of from about 0.01 to about 0.1 equivalents per equivalent of halobenzoate II.

The solvent employed in Step V is aprotic solvent V. Suitable aprotic solvents include nitriles, ethers, tertiary amides, tertiary amines, aliphatic hydrocarbons, aromatic hydrocarbons, and dialkylsulfoxides. Nitrile, ether, aliphatic hydrocarbon, and aromatic hydrocarbon solvents have been described in the discussion of previous process steps. This earlier discussion is applicable here and accordingly is incorporated herein by reference. Tertiary amide, tertiary amine and dialkylsulfoxide solvents have not been previously described. Suitable tertiary amide solvents include N,N-di-C₁-C₆ alkyl tertiary amides of C₁-C₆ alkylcarboxylic acids. Exemplary tertiary amide solvents include DMF and DMAC. Suitable tertiary amines include tri-(C₁-C₆ alkyl)amines and N—C₁-C₆ alkyl-cyclic amines. Exemplary tertiary amine solvents include TEA, DIPEA, N-methylpiperidine, and N-methylpyrrolidine. Other tertiary amine solvents suitable for use in Step V are NMM and NMP. A suitable dialkylsulfoxide solvent is DMSO.

Step V can be conducted at any temperature at which the reaction (cyanation) to form benzonitrile III can be detected. The temperature is suitably in a range of from about 60 to about 200° C., and the reaction is typically conducted at a temperature in a range of from about 80 to about 150° C. (e.g., from about 90 to about 150° C.).

The reaction (cyanation) of Step V can be conducted by charging the aprotic solvent V, the cyanide compound (plus the Pd compound and the activating ligand, as appropriate), and halobenzoate II to a suitable reaction vessel, bringing the resulting mixture to reaction temperature, and maintaining the mixture at reaction temperature (optionally with agitation such as stirring) until the reaction is complete or the desired degree of conversion of the reactants is achieved. The order of addition of the reactants and reagents to the reaction vessel is typically not critical; i.e., they can be charged concurrently or sequentially in any order. In one embodiment, halobenzoate II is first dissolved in aprotic solvent V, and the resulting solution charged to the reaction vessel, followed by addition of the cyanide compound slurried in another portion of solvent V. The reaction time can vary widely depending upon, inter alia, the reaction temperature and the choice and relative amounts of reactants, but the reaction time is typically in a range of from about 1 to about 24 hours. The benzonitrile III product can subsequently be isolated from the reaction mixture using conventional recovery procedures. In some cases the reaction mixture, after washing, filtration, and/or other treatment(s) to remove byproducts and/or unreacted substances, can be used directly in Step W. In other instances, the reaction mixture can be solvent switched (e.g., from a tertiary amide to an alcohol) for use in Step W.

The present invention includes a process for preparing a benzamide compound of Formula (VII) which comprises Steps V, W, X, Y and Z as described above and which further comprises:

(U) esterifying a benzoic acid of Formula (I):

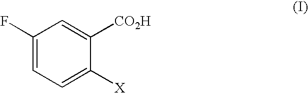

with an alcohol of formula R³—OH optionally in the presence of an acid U to obtain the halobenzoate compound of Formula (II). R³ is as defined above. Any and all embodiments and aspects of the definition of R³ set forth above in the discussion of Steps Y and Z apply here as well, and thereby provide embodiments and aspects of the definition of R³—OH and thusly embodiments and aspects of Step U and succeeding steps of the process of the invention. The alcohol R³—OH can be employed as the solvent as well as the reactant in Step U. When a separate solvent U is employed, the solvent can suitably be selected from the group consisting of aromatic hydrocarbons, halogenated aliphatic hydrocarbons, ethers and nitriles. These solvents have been described in the discussion of at least one previous process step, and the earlier discussion is applicable here and is accordingly incorporated herein by reference.

The R³—OH alcohol is typically employed in an amount that will provide for an optimum conversion and yield of benzoic acid I and halobenzoate II respectively, and is suitably employed in an amount of at least about one equivalent (e.g., from about 1 to about 20 equivalents or from about 1.5 to about 10 equivalents) of alcohol per equivalent of benzoic acid I. When the alcohol performs the dual role of reactant and solvent, the alcohol is in essence automatically employed in an amount substantially in excess of that required to react with all of the benzoic acid.

The acid U acts as a catalyst for the esterification reaction and is suitably employed in an amount in a range of from about 0.05 to about 50 equivalents per equivalent of benzoic acid I. The acid U is typically employed in an amount in a range of from about 0.05 to about 20 (e.g., from about 0.1 to about 5 or from about 0.1 to about 2) equivalents per equivalent of benzoic acid I. Acids suitable for use as acid U include the protonic acids described earlier in the discussion of Steps W and Z, including sulfuric acid, HCl, HBr, alkylsulfonic acids, arylsulfonic acids, nitric acid, and triflic acid.

Step U can be conducted at any temperature at which the reaction (esterification) to form halobenzoate II can be detected. The temperature is suitably in a range of from about 20 to about 100° C. (e.g., from about 25 to about 90° C.), and the reaction is typically conducted at a temperature in a range of from about 30 to about 80° C. (e.g., from about 40 to about 80° C.). In one embodiment, the reaction is conducted at the reflux temperature of the reaction mixture.

The esterification of Step U can be conducted by charging the alcohol reactant, optional solvent U, and benzoic acid I to a suitable reaction vessel, bringing the resulting mixture to reaction temperature, and maintaining the mixture at reaction temperature (optionally with agitation such as stirring) until the reaction is complete or the desired degree of conversion of the reactants is achieved. By-product water is typically removed (e.g., via molecular sieves) to favor formation of the desired ester product. Alternatively the reaction can be conducted at reflux temperature in the presence of the trialkyl orthoformate of formula (R³—O)₃CH corresponding to the R³—OH alcohol reactant with concurrent removal (e.g., by distillation) of alkyl formate by-product to favor formation of the desired ester. The order of addition of the reactants and reagents to the reaction vessel is typically not critical; i.e., they can be charged concurrently or sequentially in any order. In one embodiment, the alcohol reactant (also serving as the solvent) is charged to the reaction vessel first, followed by addition of benzoic acid I and the orthoformate, and then by addition of the acid U. The reaction time can vary widely depending upon, inter alia, the reaction temperature and the choice and relative amounts of reactants, but the reaction time is typically in a range of from about 1 to about 24 hours. The halobenzoate II product can subsequently be isolated from the reaction mixture using conventional recovery procedures, such as by adjusting the reaction mixture to neutral pH by addition of an aqueous solution of base, and then separating, washing, and concentrating the organic layer.

The present invention also includes a process for preparing a benzamide compound of Formula (VII) which comprises Steps Y and Z as described above wherein P* is BOC, ALLOC, or CBZ; and wherein the process further comprises:

(XA) hydrogenating a benzonitrile of Formula (III):

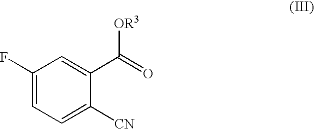

in a solvent XA, in the presence of (i) (BOC)₂O, (ALLOC)₂O, or (CBZ)₂O and (ii) Raney nickel, and optionally in the presence of a base to obtain a benzoate compound of Formula (V):

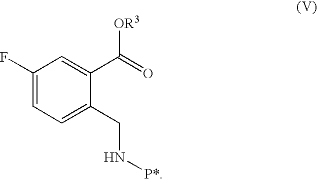

Solvent XA can suitably be selected from the group consisting of alcohols, ethers, and esters. Further description of these solvent classes is set forth above in the discussion of other process steps, is applicable here, and is incorporated herein by reference. In one embodiment, solvent XA is an ether. In an aspect of this embodiment, solvent W is a dialkyl ether wherein each alkyl is independently a C₁-C₆ alkyl, a C₁-C₆ linear or branched alkane substituted with two —O—C₁-C₆ alkyl groups (which are the same or different), or a C₄-C₈ cyclic ether or diether. In another aspect of this embodiment, solvent W is THF, dioxane, DME, MTBE, diethyl ether, or di-n-butyl ether.

The hydrogenation of the benzonitrile III can be conducted over a wide range of temperatures, although the temperature is typically in the range of from about 0 to about 100° C. (e.g., from about 10 to about 100° C.). In one embodiment, the temperature is in the range of from about 20 to about 80° C. In another embodiment, the temperature is from about 25 to about 60° C.

The pressure is not a critical aspect in Step XA, although atmospheric and superatmospheric pressures tend to be expedient. In one embodiment, the pressure is at least about 2 psig (115 kPa). In another embodiment, the pressure is in the range of from about 10 psig (170 kPa) to about 1,000 psig (6996 kPa).

The hydrogen source is typically hydrogen gas, optionally in admixture with a carrier gas that is inert under the conditions employed in Step XA (e.g., nitrogen or a noble gas such as helium or argon).

The hydrogenation in Step XA is typically conducted in the presence of a base (i.e., under basic conditions), because it has been observed that conducting the hydrogenation under basic conditions (versus acidic conditions) can result in a reduction of the amount of dimer byproduct. Suitable bases include alkali metal carbonates (Na₂CO₃ or K₂CO₃), bicarbonates (NaHCO₃ or KHCO₃), tertiary alkyl amines (TEA), tertiary cyclic amines (NMM or NMP), and pyridines. The base is suitably employed in an amount in a range of from about 0 to about 50 (e.g., from about 0.1 to about 50) equivalents per equivalent of benzonitrile III, and is typically employed in an amount in a range of from about 0.1 to about 20 (e.g., from about 0.25 to about 2 or from about 0.5 to about 1.5) equivalents per equivalent of benzonitrile III.

The amine protecting agent (i.e., (BOC)₂O, (ALLOC)₂O, or (CBZ)₂O) is typically employed in an amount sufficient to provide for the complete conversion of benzonitrile III. The amine protecting agent is suitably employed in an amount in a range of from about 1 to about 30 equivalents per equivalent of benzonitrile, and is typically employed in an amount in a range of from about 1 to 10 (e.g., from about 1 to about 5 or from about 1 to about 2) equivalents per equivalent of benzonitrile III.

The Raney Ni catalyst is suitably employed in Step XA in an amount in a range of from about 0.001 to about 1 equivalent per equivalent of benzonitrile III, and is typically employed in an amount in a range of from about 0.01 to about 0.8 equivalent per equivalent of benzonitrile III.

The uptake of hydrogen is not a critical process parameter, although at least a stoichiometric amount of hydrogen gas is typically employed.

The hydrogenation can be carried out in a pressurized reactor (e.g., an autoclave equipped with a stirrer or rocker to agitate the mixture) in which the mixture of gas (i.e., hydrogen optionally mixed with an inert gas), solvent XA, benzonitrile III, Raney nickel catalyst, amine protecting agent, and (optionally) base is continuously agitated. The order of addition of benzonitrile III, solvent, catalyst, protecting agent and base to the reaction vessel is not critical. The reactants and reagents can, for example, be added concurrently, either together or separately, or they can be added sequentially in any order. In one embodiment, benzonitrile III pre-mixed with the solvent is charged to the reaction vessel followed by addition of the protecting agent (e.g., (BOC)₂O), Raney Ni, and base. The hydrogenation can then be conducted by charging hydrogen gas, optionally in admixture with one or more inert gases, to the vessel, and then agitating the mixture under reaction conditions. The reaction time can vary widely depending upon, inter alia, the reaction temperature and pressure, the choice and relative amounts of catalyst, amine protecting agent and benzonitrile reactant, but the reaction time is typically in a range of from about 1 to about 48 hours.

The present invention also includes a process for preparing a benzamide compound of Formula (VII) which comprises Steps XA, Y and Z as described above wherein P* is BOC, ALLOC, or CBZ; and wherein the process further comprises Step V for preparing a benzonitrile of Formula (III) from a halobenzoate of Formula (II); and optionally further comprises Step U for preparing halobenzoate II from a benzoic acid of Formula (I).

The present invention also includes a process for preparing Compound 7:

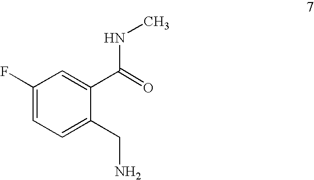

which comprises:

(yy) reacting a benzoate compound of Formula (Va):

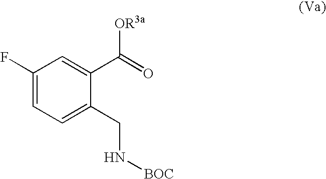

with methylamine in a solvent yy to obtain Compound 6:

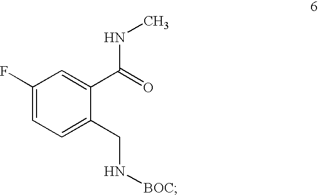

and

(zz) treating the Compound 6 with an acid zz to obtain the Compound 7; wherein R^{3a} is —C₁₋₆ alkyl.

An embodiment of this process is the process as just described, wherein the benzoate compound of Formula (Va) is Compound 5:

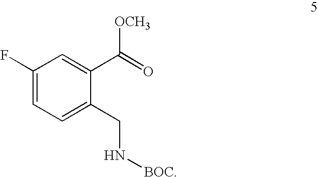

Additional embodiments of the process comprising Steps yy and zz include the process as originally set forth or as set forth in the preceding embodiment incorporating any one or more of the following aspects:

(yy-i) the reaction in Step yy is conducted at a temperature in the range of from about 50 to about 200° C. (e.g., from about 75 to about 150° C., or from about 75 to about 150° C.);

(yy-ii) methylamine is employed in Step yy in an amount in a range of from about 1 to about 200 (e.g., from about 1 to about 50, from about 1 to about 10, from about 1 to about 5, from about 1.5 to about 5, or from about 2 to about 5) equivalents per equivalent of Compound Va;

(yy-iii) the solvent yy is selected from the group consisting of alcohols, ethers, and aromatic hydrocarbons (e.g., solvent yy is selected from methanol, ethanol, n-propanol, isopropanol, n-butanol, xylene (single or mixed isomers), toluene, diethyl ether, TBF, DME and dioxane);

(zz-i) the acid zz is a mineral acid, a Lewis acid, a carboxylic acid, an alkylsulfonic acid, or an arylsulfonic acid (e.g., the acid is HCl);

(zz-ii) the acid zz is employed in Step zz in an amount in a range of from about 0.1 to about 100 (from about 0.5 to about 50, from about 1 to about 50, from about 1 to about 15, from about 1 to about 10, or from about 3 to about 15) equivalents per equivalent of Compound 6; or

(zz-iii) the treatment in Step zz is conducted in a solvent zz which is a C₁₋₆ alkyl ester of a C₁₋₆ alkylcarboxylic acid (e.g., solvent zz is C₁₋₄ alkyl ester of a C₁₋₄ alkylcarboxylic acid, and is especially a C₁₋₄ alkyl acetate such as methyl, ethyl, n-propyl, or isopropyl acetate).

The present invention includes a process for preparing Compound 7 which comprises Steps yy and zz as described above and which further comprises:

(xx) treating a benzoate compound of Formula (IVa):

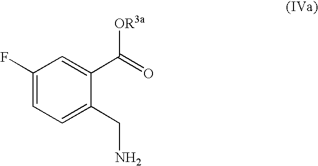

with an amine protecting agent containing the BOC group in a solvent xx to obtain the benzoate compound of Formula (Va).

In an embodiment of this process, the benzoate compound of Formula (IVa) is Compound 4:

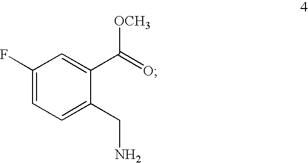

and the benzoate compound of Formula (Va) is Compound 5.

Additional embodiments include processes comprising Steps xx, yy and zz, wherein Step xx is as originally set forth or as set forth in the preceding embodiment, and wherein either Step yy incorporates one or more of aspects (yy-i) to (yy-iii) or Step zz incorporates one or more of aspects (zz-i) to (zz-iii), or both Steps yy and zz incorporate one or more of aspects thereof.

Additional embodiments of the process include processes comprising Step yy optionally incorporating any one or more of aspects (yy-i) to (yy-iii), Step zz optionally incorporating any one or more of aspects (zz-i) to (zz-iii), and Step xx as originally set forth or as set forth in the next to preceding paragraph incorporating any one or more of the following aspects:

(xx-i) the amine protecting agent in Step xx is selected from the group consisting of BOC halides and (BOC)₂O;

(xx-ii) the solvent xx is selected from the group consisting of aromatic hydrocarbons, esters, and ethers (e.g., the solvent xx is toluene, xylene (single or mixed isomers), EtOAc, IPAc, isobutyl acetate, n-butyl acetate, THF, di-n-butyl ether, dioxane, or MTBE);

(xx-iii) the treatment in Step xx is conducted at a temperature in a range of from about −20 to about 60° C. (e.g., from about −20 to about 50° C., or from about −5 to about 35° C.); or

(xx-iv) the amine protecting agent is employed in an amount in a range of from about 0.9 to about 10 (e.g., from about 0.9 to about 3 or from about 1.1 to about 3) equivalents per equivalent of benzoate compound (IVa).

The present invention includes a process for preparing Compound 7 which comprises Steps xx, yy and zz as described above and which further comprises:

(ww) hydrogenating a benzonitrile of Formula (IIIa):

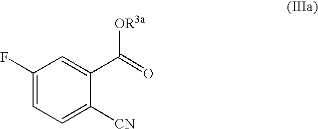

in a solvent ww and in the presence of a catalyst to obtain the benzoate compound of Formula (IVa).

In an embodiment of this process, the benzonitrile compound of Formula (IIIa) is Compound 3:

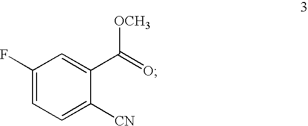

and the benzoate compound of Formula (IVa) is Compound 4.

Additional embodiments include processes comprising Steps ww, xx, yy and zz, wherein Step ww is as originally set forth or as set forth in the preceding embodiment, and wherein at least one of Steps xx, yy and zz incorporates one or more of aspects (xx-i) to (xx-iv), (yy-i) to (yy-iii), or (zz-i) to (zz-iii) respectively.

Additional embodiments of the process include processes comprising Step xx optionally incorporating any one or more of aspects (xx-i) to (xx-iv), Step yy optionally incorporating any one or more of aspects (yy-i) to (yy-iii), Step zz optionally incorporating any one or more of aspects (zz-i) to (zz-iii), and Step ww as originally set forth or as set forth in the next to preceding paragraph incorporating any one or more of the following aspects:

(ww-i) the catalyst employed in the hydrogenation in Step ww is supported or unsupported and is selected from the group consisting of Pd, Pt, and compounds thereof;

(ww-ii) the hydrogenation in Step ww is conducted under acidic conditions in the presence of a protonic acid ww (e.g., the protonic acid ww is HCl);

(ww-iii) the hydrogenation in Step ww is conducted at a temperature in a range of from about 0 to about 100° C. (e.g., from about 10 to 100° C., from about 15 to about 60° C., or from about 25 to about 45° C.);

(ww-iv) the catalyst is employed in an amount in a range of from about 0.001 to 1 equivalent (e.g., from about 0.01 to about 0.8, from about 0.02 to about 0.5, from about 0.02 to about 0.10, or from about 0.02 to about 0.8) equivalents per equivalent of the benzonitrile of Formula (IIIa); or

(ww-v) the solvent ww is an alcohol (e.g., solvent ww is a C₁-C₄ alkyl alcohol, such as methanol, ethanol, n-propanol, isopropanol, or isobutanol).

The present invention includes a process for preparing Compound 7 which comprises Steps ww, xx, yy and zz as described above and which further comprises:

(vv) reacting a halobenzoate compound of Formula (IIa):

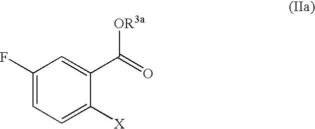

in an aprotic solvent vv with a cyanide compound selected from the group consisting of CuCN and Zn(CN)₂ to obtain the benzonitrile of Formula (IIIa); with the proviso that when the cyanide compound is Zn(CN)₂, the reaction is conducted in the presence of a Pd compound and an activating ligand; wherein X is chloro, bromo, or iodo.

In an embodiment of this process, the halobenzoate compound of Formula (IIa) is Compound 2:

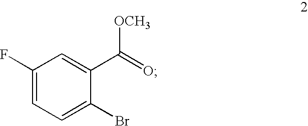

and the benzonitrile compound of Formula (IIIa) is Compound 3.

Additional embodiments include processes comprising Steps vv, ww, xx, yy and zz, wherein Step vv is as originally set forth or as set forth in the preceding embodiment, and wherein at least one of Steps ww, xx, yy and zz incorporates one or more of aspects (ww-i) to (ww-v), (xx-i) to (xx-iv), (yy-i) to (yy-iii), or (zz-i) to (zz-iii) respectively.

Additional embodiments of the process include processes comprising Step ww optionally incorporating any one or more of aspects (ww-i) to (ww-v), Step xx optionally incorporating any one or more of aspects (xx-i) to (xx-iv), Step yy optionally incorporating any one or more of aspects (yy-i) to (yy-iii), Step zz optionally incorporating any one or more of aspects (zz-i) to (zz-iii), and Step vv as originally set forth or as set forth in the next to preceding paragraph incorporating any one or more of the following aspects:

(vv-i) the aprotic solvent vv is a tertiary amide (e.g., the solvent is DMF or DMAC);

(vv-ii) the reaction in Step vv is conducted at a temperature in a range of from about 60 to about 200° C. (e.g., from about 80 to about 150° C. or from about 90 to about 150° C.);

(vv-iii) the cyanide compound is CuCN; or

(vv-iv) the cyanide compound (e.g., CuCN) is employed in Step vv in an amount in a range of from about 0.5 to about 30 (e.g., from about 0.5 to about 10, from about 0.5 to about 5, from about 0.9 to about 2, or from about 0.9 to about 1.5) equivalents per equivalent of the halobenzoate compound of Formula (IIa).

The present invention includes a process for preparing Compound 7 which comprises Steps vv, ww, xx, yy and zz as described above and which further comprises:

(uu) esterifying a benzoic acid of Formula (I):

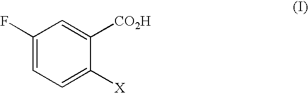

with an alcohol of formula R^{3a}—OH optionally in the presence of an acid uu to obtain the halobenzoate compound of Formula (IIa).

In an embodiment of this process, the benzoic acid of Formula (I) is Compound 1:

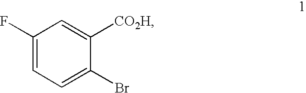

and the halobenzoate compound of Formula (IIa) is Compound 2.

Additional embodiments include processes comprising Steps uu, vv, ww, xx, yy and zz, wherein Step vv is as originally set forth or as set forth in the preceding embodiment, and wherein at least one of Steps vv, ww, xx, yy and zz incorporates one or more of aspects (vv-i) to (vv-iv), (ww-i) to (ww-v), (xx-i) to (xx-iv), (yy-i) to (yy-iii), or (zz-i) to (zz-iii) respectively.

Additional embodiments of the process include processes comprising Step vv optionally incorporating any one or more of aspects (vv-i) to (vv-iv), Step ww optionally incorporating any one or more of aspects (ww-i) to (ww-v), Step xx optionally incorporating any one or more of aspects (xx-i) to (xx-iv), Step yy optionally incorporating any one or more of aspects (yy-i) to (yy-iii), Step zz optionally incorporating any one or more of aspects (zz-i) to (zz-iii), and Step uu as originally set forth or as set forth in the next to preceding paragraph incorporating any one or more of the following aspects:

(uu-i) the alcohol of formula R^{3a}—OH acts as the solvent for Step uu (and thus is present in an amount substantially in excess of that which is required to react with benzoic acid I);

(uu-ii) the acid uu is employed in an amount in a range of from about 0.05 to about 50 (e.g., from about 0.05 to about 20, from about 0.1 to about 5, or from about 0.1 to about 2) equivalents per equivalent of benzoic acid I;

(uu-iii) the acid uu is a mineral acid (e.g., sulfuric acid); or

(uu-iv) the esterification in Step uu is conducted at a temperature in a range of from about 20 to about 100° C. (e.g., from about 25 to about 90° C., from about 30 to about 80° C., or from about 40 to about 80° C.).

The present invention includes a process for preparing Compound 7 which comprises Steps yy and zz as described above and which further comprises:

(xxa) hydrogenating a benzonitrile of Formula (IIIa):

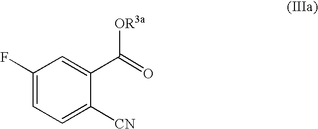

in a solvent xxa, in the presence of (BOC)₂O and Raney nickel, and optionally in the presence of a base to obtain a benzoate compound of Formula (Va):

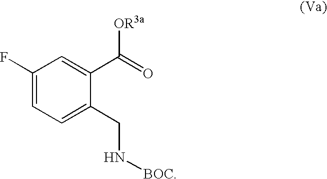

In an embodiment of this process, the benzonitrile of Formula (IIIa) is Compound 3; and the benzoate compound of Formula (Va) is Compound 5.

Additional embodiments include processes comprising Steps xxa, yy and zz, wherein Step xxa is as originally set forth or as set forth in the preceding embodiment, and wherein at least one of Steps yy and zz incorporates one or more of aspects (yy-i) to (yy-iii) or (zz-i) to (zz-iii) respectively.

Additional embodiments of the process include processes comprising Step yy optionally incorporating any one or more of aspects (yy-i) to (yy-iii), Step zz optionally incorporating any one or more of aspects (zz-i) to (zz-iii), and Step xxa as originally set forth or as set forth in the preceding paragraph incorporating any one or more of the following aspects:

(xxa-i) the hydrogenation in Step xxa is conducted at a temperature in a range of from about 0 to about 100° C. (e.g., from about 10 to about 100° C., from about 20 to about 80° C., or from about 25 to about 60° C.);

(xxa-ii) (BOC)₂O is employed in Step xxa in an amount in a range of from about 1 to about 30 (e.g., from about 1 to about 10, from about 1 to about 5, or from about 1 to about 2) equivalents per equivalent of the benzonitrile IIIa;

(xxa-iii) solvent xxa is selected from the group consisting of ethers and esters (e.g., solvent xxa is an ether, such as THF, dioxane, DME, MTBE, or di-n-butyl ether);

(xxa-iv) the optional base in Step xxa is an alkali metal bicarbonate (e.g., NaHCO₃ or KHCO₃);

(xxa-v) the amount of base employed in Step xxa is in a range of from about 0.1 to about 20 (from about 0.25 to about 2, from about 0.5 to about 1.5, or from about 1 to about 2) equivalents per equivalent of the benzonitrile IIIa; or

(xxa-vi) Raney nickel is employed in an amount in a range of from about 0.001 to about 1 (e.g., from about 0.01 to about 0.8) equivalent per equivalent of benzonitrile IIIa.

The present invention also includes a process for preparing Compound 7 which comprises Steps xxa, yy, and zz as described above wherein the process further comprises Step vv for preparing a benzonitrile of Formula (IIIa) from a halobenzoate of Formula (IIa); and optionally further comprises Step uu for preparing halobenzoate IIa from a benzoic acid of Formula I.

The processes for preparing Compound 7 (i.e., those processes involving Steps xx and yy and optionally one or more other steps as described above) can be conducted using procedures the same or similar to those described earlier for the analogous processes for preparing Compound VII (i.e., those processes involving Steps X and Y and optionally one or more other steps as earlier described).

Still other embodiments of the present invention include any of the processes as originally defined and described above and any embodiments or aspects thereof as heretofore defined, further comprising isolating (which may be alternatively referred to as recovering) the compound of interest (e.g., Compound VII or Compound 7, in the form of an acid salt or as the free base) from the reaction medium.

The progress of any of the above-described reaction steps (i.e., Steps U, V, W, X, XA, Y and Z or Steps uu, vv, ww, xx, xxa, yy and zz) can be followed by monitoring the disappearance of a reactant (e.g., Compound V in Step Y) and/or the appearance of the product (e.g., Compound VII in Step Z) using such analytical techniques as TLC, HPLC, IR, NMR or GC.

The present invention also includes a compound of Formula (IIIa), a compound of Formula (IVa) (or a salt thereof), and a compound of Formula (Va), all as defined and described above.

The present invention also includes Compound 3, Compound 4 (or a salt thereof), and Compound 5, all as set forth above.

As used herein, the term “C₁₋₆ alkyl” (or “C₁-C₆ alkyl”) refers to a linear or branched chain alkyl group having from 1 to 6 carbon atoms and includes all of the hexyl alkyl and pentyl alkyl isomers as well as n-, iso-, sec- and t-butyl, n- and isopropyl, ethyl and methyl. “C₁₋₄ alkyl” refers to n-, iso-, sec- and t-butyl, n- and isopropyl, ethyl and methyl. “C₁₋₃ alkyl” refers to n- and isopropyl, ethyl and methyl.

The term “C₃₋₆ cycloalkyl” (or “C₃-C₆ cycloalkyl”) means a cyclic ring of an alkane having three to six total carbon atoms (i.e., cyclopropyl, cyclobutyl, cyclopentyl, or cyclohexyl).

The term “C₁₋₆ alkyloxy” refers to a —O—C₁₋₆ alkyl in which the C₁₋₆ alkyl group is as defined above. “C₁₋₄ alkyloxy” has an analogous meaning.

The term “halogen” (or “halo”) refers to fluorine, chlorine, bromine and iodine (alternatively referred to as fluoro, chloro, bromo, and iodo).

The term “aryl” as used herein refers to an aromatic carbocyclic ring or an aromatic carbocyclic fused ring system. The fused ring system contains two or more carbocyclic rings in which each ring shares two adjacent carbon atoms with at least one other ring. The aryl group may be attached to the rest of the molecule at any carbon atom which results in a stable compound. A subset of aryl groups particularly suitable for use in the present invention includes those selected from phenyl, naphthyl, anthryl (also referred to as “anthracenyl”), and phenanthryl (or “phenanthrenyl”). Another particularly suitable subset of aryl groups is phenyl and naphthyl. Still another particularly suitable subset of aryl groups is phenyl per se.

When any variable (e.g., R^{a}, R^{b}, or R^{c}) occurs more than one time in Formulas V to VII or in any other formula depicting and describing compounds employed in the process of the invention, its definition on each occurrence is independent of its definition at every other occurrence. Also, combinations of substituents and/or variables are permissible only if such combinations result in stable compounds.

The term “substituted” (e.g., as in “aryl, optionally substituted with from 1 to 6 substituents . . . ”) includes mono- and poly-substitution by a named substituent to the extent such single and multiple substitution (including multiple substitution at the same site) is chemically allowed.

The term “solvent” in reference to the solvent employed in a process step (e.g., solvents Y and Z employed in Steps Y and Z respectively) can be any organic compound which under the reaction conditions employed is in the liquid phase, is chemically inert (unless expressly stated to the contrary; e.g., the alcohol in Step U can be both reactant and solvent), and will dissolve, suspend, and/or disperse the reactants so as to bring the reactants into contact and permit the reaction to proceed.

Unless expressly stated to the contrary, any range (e.g., a temperature range) cited herein is inclusive; i.e., the range includes the values for the upper and lower limits of the range as well as all values in between.

Abbreviations used in the instant specification include the following:

Ac=acetyl

ALLOC=allyloxycarbonyl

BINAP=2,2′-bis(diphenylphosphino)-1,1′-binaphthyl

Bn=benzyl

BOC or Boc=t-butyloxycarbonyl

(BOC)₂O (or BOC₂O)=di-t-butyl carbonate

CBZ or Cbz=carbobenzoxy (alternatively, benzyloxycarbonyl)

dba=dibenzylideneacetone

DCE=1,2-dichloroethane

DIPEA=diisopropylethylamine (or Hunig's base)

DMAC=N,N-dimethylacetamide

DME=1,2-dimethoxyethane

DMF=N,N-dimethylformamide

dppb=1,4-bis(diphenylphosphino)butane

dppe=1,2-bis(diphenylphosphino)ethane

dppp=1,3-bis(diphenylphosphino)propane

dppf=diphenylphosphinoferrocene

EDC=1-ethyl-3-(3-dimethylaminopropyl) carbodiimide

EtOAc=ethyl acetate

HPLC=high performance liquid chromatography

IPAc=isopropyl acetate

IR=infrared spectroscopy

KF=Karl Fisher titration for water

Me=methyl

MeOH=methanol

MTBE=methyl tert-butyl ether

NBS=N-bromonsuccinimide

NMM=N-methylmorpholine

NMP=N-methyl pyrrolidinone

NMR=nuclear magnetic resonance

Ph=phenyl

Pr=propyl

i-Pr=isopropyl

TEA=triethylamine

THF=tetrahydrofuran

TLC=thin layer chromatography

### EXAMPLE 1

### Potassium 5-(1,1-dioxido-1,2-thiazinan-2-yl)-7-[({4-fluoro-2-[(methylamino)-carbonyl]benzyl}amino)carbonyl]-1,6-naphthyridin-8-olate

Step 1: Methyl 2-bromo-5-fluorobenzoate

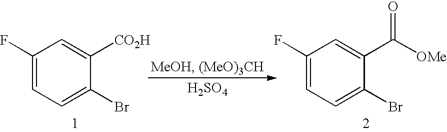

| Material | MW | Amount | Moles |
|---|---|---|---|
| 2-bromo-5-fluorobenzoic acid | 219.01 | 4.00 kg | 18.3 |
| methanol | 32.04 | 18 L | 296.3 |
| | (d = 0.791) | | |
| trimethylorthoformate | 106.12 | 3.88 kg | 36.5 |
| 96% sulfuric acid | 98.08 | 0.373 kg | 3.65 |
| 2 M K₂HPO₄ | 174.18 | 4.82 L | 9.68 |
| ethyl acetate | | 16 L | |
| 10% NaHCO₃ | 84.02 | 4 L | |
| 25% brine | | 4 L | |
| toluene | | 12 L | |
| DMF | | | |

To a 72 L round bottom flask, equipped with an overhead stirrer, thermocouple, water-cooled condenser, and nitrogen inlet, was charged methanol (18 L). 2-Bromo-5-fluorobenzoic acid (4.00 kg), trimethyl orthoformate (3.876 kg), were then charged with stirring, followed by the addition 96% sulfuric acid (0.373 kg). The resulting solution was refluxed at 63° C. and aged for 10-16 hr, while the by-product (methyl formate) was removed during the reaction. The reaction mixture was monitored by HPLC (conversion was >99%). The reaction mixture was concentrated, then diluted with ethyl acetate (16 L), and cooled to 20° C. 2 M potassium hydrogen phosphate (4.82 L) was then added to adjust the pH to 6.5-7. The mixture was then transferred to a 100 L nalgene extractor. After phase cut, the organic layer was washed with 10% NaHCO₃ (4 L), 25% brine (4 L), and then concentrated under reduced pressure. The residual oil was dissolved in toluene (6 L), and concentrated. This operation was done one more time. The remaining oil was dissolved in DMF (total vol. 9.2 L). The resulting solution was used for next step.

HPLC conditions: column: Zorbax, Rx C_{8 250×4.6} mm; temperature: 30° C.; detection: 210 nm; mobile phase: 0.1% aq H₃PO₄ (A)/MeCN (B); gradient: 90:10 (A)/(B) to 10:90 over 15 min, 10:90 hold for 5 min, 10:90 to 90:10 (A)/(B) over 10 seconds; flow rate: 1 mL/min; retention time for the desired monoester; 13.6 min.

Evaporation of a sample to dryness gave a colorless oil: ¹H NMR (400 MHz, CDCl₃) δ: 7.64 (dd, J=8.8, 5.0 Hz, 1H), 7.53 (dd, J=8.8, 3.1 Hz, 1H), 7.08 (td, J=8.8, 3.1 Hz, 1H), 3.95 (s, 3H); ¹³C NMR (100 MHz, CDCl₃) δ: 165.4, 161.3 (d, J=240.0 Hz), 135.9, 133.4, 120.0 (d, J=20.0 Hz), 118.5 (d, J=20.0 Hz), 116.1, 52.7.

Step 2: Methyl 2-nitrile-5-fluorobenzoate

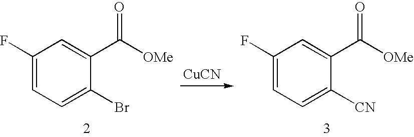

| Material | MW | Amount | Moles |
|---|---|---|---|
| methyl 2-bromo-5-fluorobenzoate | 233.03 | | 18.3 in DMF |
| copper(I) cyanide | 89.56 | 1.60 kg | 17.9 |
| DMF | | 5 L + 4 L | |
| ethyl acetate | | 35 L + 17 L | |
| 10% NH₄OH-20% NH₄Cl | | 37 L | |
| 25% brine | | 8 L | |
| MeOH | | 33 L | |

To a solution of methyl 2-bromo-5-fluorobenzoate (18.26 moles) in DMF (total vol. 9.2 L) was charged copper(I) cyanide (1.603 kg) in DMF (5 L) slurry and followed with a DMF flush (4 L). After being degassed, the reaction mixture was heated at 100° C. for 10-16 hours. The reaction mixture was monitored by HPLC (conversion was >98%). After being cooled to 50° C.-60° C., ethyl acetate (20 L) was added, and then 10% NH₄OH-20% NH₄Cl (22 L). The mixture was then transferred to a 100 L nalgene extractor. The 72 L round bottom flask was washed with 15 L of EtOAc and 15 L of water and transferred to the 100 L extractor. After phase cut, the aqueous layer was back-extracted with EtOAc (17 L) one time. The combined organic layers were washed with 10% NH₄OH/20% NH₄Cl:water (1:1, 3×10 L), 16% brine (8 L), concentrated, and solvent switched to MeOH (total vol. 22 L, KF=152.6 μg/mL). The resulting solution was used for next step.

HPLC conditions: column: Zorbax, Rx C_{8 250×4.6} mm; temperature: 30° C.; detection at 210 nm; mobile phase: 0.1% aq H₃PO₄ (A)/MeCN (B); gradient: 90:10 (A)/(B) to 10:90 over 15 min, 10:90 hold for 5 min, 10:90 to 90:10 (A)/(B) over 10 seconds; flow rate: 1 mL/min; retention time for the desired monoester: 11.7 min.

Evaporation of a sample to dryness gave a light yellow solid: ¹H NMR (CDCl₃) δ: 7.86-7.80 (m, 2H), 7.37 (td, J=8.5, 2.6 H, 1H), 4.02 (s, 3H); ¹³C NMR (100 MHz, CDCl₃) δ: 164.3 (d, J=260 Hz), 163.3, 137.1 (d, J=10.0 Hz), 135.2 (d, J=10.0 HZ), 120.2 (d, J=30.0 Hz), 118.8 (d, J=20.0 Hz), 116.6, 109.0, 53.1.

Step 3: Methyl 2-aminomethyl-5-fluorobenzoate, HCl salt

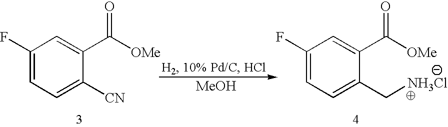

| Material | MW | Amount | Moles |
|---|---|---|---|
| methyl 2-nitrile-5-fluorobenzoate | 179.15 | | 10.6 in MeOH |
| 3.0 M HCl in MeOH (anhydrous) | 36.46 | 7.10 L | 21.22 |
| 10% Pd/C | | 0.475 kg | |
| solka floc | | 2.6 kg | |
| MeOH | | 3 × 10 L | |

A degassed mixture of methyl 2-nitrile-5-fluorobenzoate (10.6 moles) in MeOH (total 10.0 L), 3.0 M HCl in MeOH (7.10 L), and 10% Pd/C (0.475 kg) was submitted to hydrogenation at 40° C. and 45 PSI for 48 hours. The reaction mixture was monitored by HPLC (conversion was >97%). After being cooled to ambient temperature, the reaction mixture was then filtered by passing a short Solka Flock (2.6 kg), which was washed with MeOH (3×10 L). The combined filtrates were concentrated and solvent-switched to toluene in total volume (about 18 L, KF=154 μg/mL). The crystalline solid was filtered off and washed with toluene, dried under vacuum with nitrogen sweep to afford 2.02 kg of the title compound (87% isolated yield overall for the three steps, >99A % purity, HPLC).

HPLC conditions: column: Zorbax, Rx C_{8 250×4.6} mm; temperature: 30° C.; detection at 210 nm; mobile phase: 0.1% aq H₃PO₄ (A)/MeCN (B); gradient: 90:10 (A)/(B) to 10:90 over 15 min, 10:90 hold for 5 min, 10:90 to 90:10 (A)/(B) over 10 seconds; flow rate: 1 mL/min; retention time for the desired monoester: 5.78 min.

¹H NMR (CDCl₃) δ: 8.43 (brs, 3H), 7.74-7.65 (m, 2H), 7.55 (td, J=8.4, 2.8 Hz, 1H), 4.26 (q, J=5.5 Hz), 3.85 (s, 3H); ¹³C NMR (100 MHz, CDCl₃) δ: 165.8, 162.1 (d, J=250 Hz), 134.8 (d, J=10.0 Hz), 131.9 (d, J=10.0 Hz), 131.7, 120.1 (d, J=20.0 Hz), 117.7 (d, J=30.0 Hz), 53.2, 40.3.

Step 4: Methyl 2-t-butyloxycarbonylaminomethyl-5-fluorobenzoate

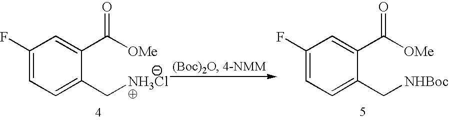

| Material | MW | Amount | Moles |
|---|---|---|---|
| ammonium salt 4 | 219.64 | 3.42 kg | 15.6 |
| (BOC)₂O | 218.25 | 3.73 kg | 17.1 |
| NMM | 101.15 (d = 0.920) | 1.73 kg | 17.1 |
| 40 wt. % MeNH₂ | 31.06 | 1.21 kg | 15.6 |
| toluene | | 31 L | |
| 0.1 M EDTA Na sol'n | | 6.2 L | |
| 25% brine | | 6.2 L | |

To the ammonium salt 4 (3.42 kg) in toluene (31 L) was added (BOC)₂O (3.73 kg), followed by NMM (1.73 kg), at 15° C.-20° C. over 1 hour. The reaction mixture was aged at room temperature for 15-24 hours (conversion as determined by HPLC was >99%), followed by the addition of 40 wt % methylamine aqueous (1.21 kg) at 5° C.-10° C., after which the mixture was aged at the same temperature for 2 hours to quench the excess (BOC)₂O. The reaction mixture was then worked up by charging water (12 L). After phase cut, the organic layer was washed with 0.1 M EDTA sodium solution (6.2 L), 25% brine (6.2 L), and concentrated to total volume (20 L), which was divided by two equal amount portions for amidation in two batches.

HPLC conditions: column: Zorbax, Rx C_{8 250×4.6} mm; temperature: 30° C.; detection at 210 nm; mobile phase: 0.1% aq H₃PO₄ (A)/MeCN (B); gradient: 90:10 (A)/(B) to 10:90 over 15 min, 10:90 hold for 5 min, 10:90 to 90:10 (A)/(B) over 10 seconds; flow rate: 1 mL/min; retention time for the desired monoester: 14.5 min.

Evaporation of a sample to dryness gave a colorless oil: ¹H NMR (CDCl₃) δ: 7.65 (dd, J=9.4, 2.4, 1H), 7.50 (dd, J=8.0, 5.7 Hz, 1H), 7.18 (dd, J=8.0, 2.8 Hz, 1H), 5.31 (brs, 1H), 4.47 (d, J=6.6 Hz, 1H), 3.91 (s, 3H), 1.41 (s, 9H); ¹³C NMR (100 MHz, CDCl₃) δ: 166.5, 1.61.5 (d, J=250 Hz), 155.8, 137.0, 132.8 (d, J=10.0 Hz), 130.2 (d, J=10.0 Hz), 119.6 (d, J=30.0 Hz), 117.7 (d, J=20.0 Hz), 79.2, 52.4, 42.9, 28.4 (3C).

Step 4a: Alternative to Steps 3-4 for the preparation of methyl 2-t-butyloxycarbonylaminomethyl-5-fluorobenzoate

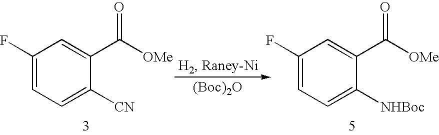

| Material | MW | Amount | Moles |
|---|---|---|---|
| methyl 2-nitrile-5-fluorobenzoate | 179.15 | | 9.13 in THF |
| (BOC)₂O | 218.25 | 2.191 kg | 10.04 |
| NaHCO3 | 84.01 | 0.8448 Kg | 10.04 |
| Raney Ni | | 0.409 Kg | |
| Solka floc | | 1 Kg | |
| THF | | 3 × 5 L | |
| Toluene | | 20 L | |
| methylamine | 31.06 | 0.284 Kg | 9.13 |

A degassed mixture of methyl 2-nitrile-5-fluorobenzoate (3, 9.13 moles), (BOC)₂O (2.191 Kg), sodium bicarbonate (0.844 Kg), and Raney-Ni (0.409 Kg, anhydrous) in THF was hydrogenated at 50° C. for 24 hours. Conversion as determined by HPLC was >99%. After cooling the reaction mixture to ambient temperature, the mixture was filtered through Solka Flock (1 Kg) which was then washed with THF (3×5 L). The combined filtrates were cooled to −20° C. and then methylamine (0.284 Kg) was added by bubbling to remove excess (BOC)₂O. The reaction mixture was stirred at ambient temperature for 1 hour, after which the mixture was concentrated and solvent switched to toluene (total vol. 25 L). The resulting solution was washed with 0.1 M EDTA disodium salt (2×5 L), 10% NaHCO₃:16% brine (1:4, 5 L), concentrated to a total volume (11 L). The solution was suitable for use in the next step.

HPLC conditions: column: Zorbax, Rx C_{8 250×4.6} mm; temperature: 30° C.; detection at 210 nm; mobile phase: 0.1% aq H₃PO₄ (A)/MeCN (B); gradient: 90:10 (A)/(B) to 10:90 over 15 min, 10:90 hold for 5 min, 10:90 to 90:10 (A)/(B) over 10 seconds; flow rate: 1 mL/min; retention time for the desired monoester; 14.45 min

Step 5: N-methyl 2-t-butyloxycarbonylaminomethyl-5-fluorobenzenecarboxamide

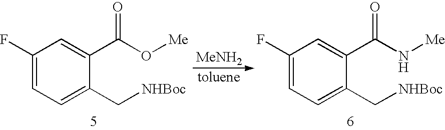

| Material | MW | Amount | Moles |
|---|---|---|---|
| methyl benzoate 5 | 283.30 | | 7.77 in toluene |
| methylamine | 31.06 | 0.483 kg | 15.6 |
| toluene | | 5 L | |
| heptane | | 50 L + 25 L | |

The crude methyl benzoate 5 in toluene (7.77 moles in 10 L) was cooled to −20° C. and methylamine (0.483 kg) gas was added. The mixture was then heated in an autoclave at 80-85° C. for 48 hours. The reaction was monitored by HPLC (conversion was >98%). After cooling to about 50° C., the reaction mixture was transferred to a large round bottom flask for batch concentration. The solution was concentrated, producing a slurry, and solvent-switched to toluene (total vol. 12 L), after which heptane (50 L) was slowly charged to the slurry. The resulting slurry was aged at 0° C. for 1 hour. The white crystalline solid was filtered off, rinsed with heptane (25 L), and dried under vacuum with a nitrogen sweep to give methylamide 6 (1.92 kg, 83% overall yield for the two preceding steps after correcting to pure product).

HPLC conditions: column: Zorbax, Rx C_{8 250×4.6} mm; temperature: 30° C.; detection at 210 nm; mobile phase: 0.1% aq H₃PO₄ (A)/MeCN (B); gradient: 90:10 (A)/(B) to 10:90 over 15 min, 10:90 hold for 5 min, 10:90 to 90:10 (A)/(B) over 10 seconds; flow rate: 1 mL/min; retention time for the desired product: 11.6 min.

¹H NMR (CDCl₃) δ: 7.43 (dd, J=8.4, 5.5 Hz, 1H), 7.15-7.07 (m, 2H), 6.52 (brs, 1H), 5.66 (brs, 1H), 4.28 (d, J=6.4 Hz, 2H), 3.10 (d, J=4.8 H, 3H), 1.42 (s, 9H); ¹³C NMR (100 MHz, CDCl₃) δ: 169.0, 161.5 (d, J=250 Hz), 156.1, 137.3, 133.5, 132.0 (d, J=10.0 Hz), 117.2 (, d, J=20.0 Hz), 114.3 (d, J=20.0 Hz), 79.4, 42.2, 26.7.

Step 6: N-methyl 2-aminomethyl-5-fluorobenzenecarboxamide, HCl salt

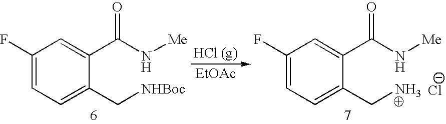

| Material | MW | Amount | Moles |
|---|---|---|---|
| N-methyl amide 6 | 282.31 | 3.14 kg | 11.1 |
| HCl (gas) | 36.46 | 3.25 kg | 89.0 |
| EtOAc | | 21.4 L + 42.8 L + 30 L | |
| heptane | | 40 L | |

HCl gas (3.25 Kg) was bubbled into ethyl acetate (21.4 L) at −20° C. N-Methyl amide 6 (3.14 kg) was charged to the HCl-EtOAc solution, and the reaction mixture was warmed to ambient temperature (17° C.) in about 3 hours and aged for 2-4 hours. The reaction was monitored by HPLC (conversion was >99%). The reaction mixture was diluted with EtOAc (42.8 L), and the resulting slurry was aged at 0-5° C. for 0.5 hour. The crystalline solid was filtered off and washed with EtOAc (30 L), then with heptane (40 L), and then dried under vacuum with a nitrogen sweep to give the salt. The crystalline solid (2.434 kg) was recrystallized by dissolved in methanol (10.5 L) at 30° C. To the resulting solution was added EtOAc (64 L), producing a slurry that was aged at 0-5° C. for 1 hour. The white crystalline solid was filtered off and washed with EtOAc (30 L), dried under vacuum with nitrogen sweep to give the desired product (2.14 kg, 91% isolated yield corrected for starting material purity; >99.5 A % purity).

HPLC conditions: column: Zorbax, Rx C_{8 250×4.6} mm; temperature: 30° C.; detection at 210 nm; mobile phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; flow rate: 1 mL/min; retention time for the desired product: 3.33 min.

¹H NMR (CDCl₃) δ: 8.84 (brs, 1H), 8.05 (brs, 3H), 7.55 (dd, J=8.3, 5.8 Hz, 1H), 7.46-7.13 (m, 2H), 4.01 (s, 3H), 2.77 (d, J=4.6 Hz, 3H); ¹³C NMR (100 MHz, CDCl₃) δ: 167.9, 162.0 (d, J=250 Hz), 157.9, 138.5 (d, J=10.0 Hz), 134.3 (d, J=10.0 Hz), 129.2, 117.6 (d, J=20.0 Hz), 115.5 (d, J=20.0 Hz), 40.7, 26.7.

Step 7: 5-(1,1-Dioxido-1,2-thiazinan-2-yl)-8-hydroxy-1,6-naphthyridine-7-carboxylic acid

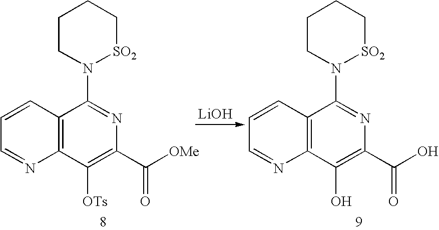

| Material | MW | Equivalents | Amount | Moles |
|---|---|---|---|---|
| Tosylate 8 | 491.5 | 1.0 | 3.3 kg | 6.7 |
| 2-propanol | | 4 L/kg 8 | 13.2 L | |
| water | | 4 L/kg 8 | 13.2 L | |
| LiOH · H₂O | 41.96 | 3.3 | 0.93 | 22.2 |
| 2 N HCl | | 2.6 | 8.7 L | 17.5 |
| Water | | 5 L/kg 8 | 4 × 4.3 L | |

A 50-L flask equipped with a mechanical stirrer, temperature probe, addition funnel, and nitrogen inlet was charged with 2-propanol (13.2 L) and tosylate 8 (3.3 kg). The lithium hydroxide monohydrate (0.93 kg) was then charged as a solution in GMP water (13.2 L) at 20-25° C. The resulting suspension was warmed to 60° C. where a homogeneous yellow solution was obtained. The reaction was aged until complete conversion to 9 was reached as determined by HPLC assay (4-16 hours). The resulting yellow suspension was cooled to about 20° C. and diluted with 2 N HCl (8.7 L) over 0.5 hour. The pH was between 1.3-1.6 at 20° C. following HCl addition. The suspension was cooled to about 20° C., filtered, and the cake was washed with water (4×4.3 L) as displacement washes. The cake was dried on the filter pot under nitrogen and house vacuum until the water content was <6 wt % by Karl Fisher titration. The purity of carboxylic acid phenol 9 was >99.4 A % by HPLC assay.

¹H NMR (DMSO-d6, 400 MHz) δ 9.21 (1H, dd, J=4.3, 1.6 Hz), 8.62 (1H, dd, J=8.5, 1.6 Hz), 7.92 (1H, dd, J=8.5, 4.3 Hz), 3.91-3.78 (2H, m), 3.55-3.45 (2H, m), 2.28 (3H, m) and 1.64 (1H, m) ppm.

Step 8: 5-(1,1-Dioxido-1,2-thiazinan-2-yl)-N-{4-fluoro-2-[(methylamino)carbonyl]benzyl}-8-hydroxy-1,6-naphthyridine-7-carboxamide

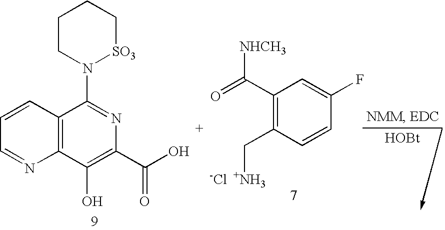

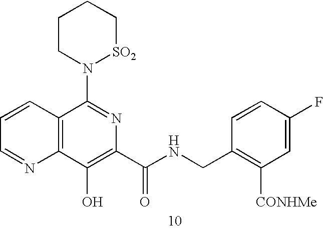

| Material | MW | Equivalents | Amount | Moles |
|---|---|---|---|---|
| carboxylic acid 9 | 323.33 | 1.0 | 1.63 kg | 5.04 |
| DMF | | 10 L/kg 9 | 16.3 L | |
| amine 7 | 218.66 | 1.2 | 1.32 kg | 6.05 |
| HOBt | 135.13 | 0.5 | 341 g | 2.52 |
| NMM | 101.15 | 0.9 | 456 g | 4.54 |
| EDC · HCl | 191.71 | 1.5 | 1.45 kg | 7.56 |
| water | | 10 L/kg 9 | 16.3 L | |

A 50-L flask equipped with a mechanical stirrer, temperature probe, and nitrogen inlet was charged with the dry DMF (16.3 L), carboxylic acid 9 (1.73 kg gross, 1.63 assay kg, KF=6.0 wt % water), anhydrous HOBt (341 g), amine 7 (1.32 kg), and NMM (456 g, 500 mL). The suspension was agitated at 20° C. until a homogeneous solution was obtained and then cooled to 0-5° C. The EDC (1.45 kg) was added and the reaction aged until complete conversion of 9 was reached as determined by HPLC (<0.5% 9, about 16 hours). The reaction was diluted with water (1.6 L) at 20° C., seeded (11 g), and aged for 0.5 hour. The batch was diluted with water (14.7 L) to give a 1:1 v/v ratio of water:DMF and then cooled to 0° C. The batch was then filtered and the cake washed with chilled 1:1 water:DMF (4×2.5 L) and chilled water (4×5.5 L) as displacement washes. The cake was then dried at ambient temperature under nitrogen tent/house vacuum to obtain the title product (2.16 kg, 88% isolated yield, purity: >99.0 A % by HPLC assay).

¹H NMR (DMSO-d6, 400 MHz) δ 9.53 (1H, s), 9.19 (1H, s), 8.68 (1H, s), 8.58 (1H, d, J=8.0 Hz), 7.89 (1H, d, J=3.8 Hz), 7.53 (1H, m), 7.41-7.34 (2H, m), 4.64 (2H, d, J=5.7 Hz), 3.92-3.47 (4H, m), 2.83 (3H, d, J=3.8 Hz), 2.35 (3H, m), and 1.64 (1H, m) ppm.

While the foregoing specification teaches the principles of the present invention, with examples provided for the purpose of illustration, the practice of the invention encompasses all of the usual variations, adaptations and/or modifications that come within the scope of the following claims.

What is claimed is:

1. A process for preparing a benzamide compound of Formula (VII): 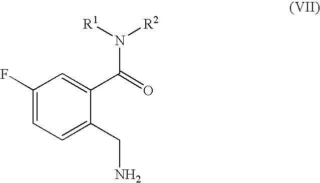
which comprises:
(Y) reacting a benzoate compound of Formula (V): 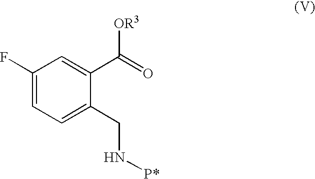
with an amine of formula R¹R²NH in a solvent Y to obtain a benzamide compound of Formula (VI): 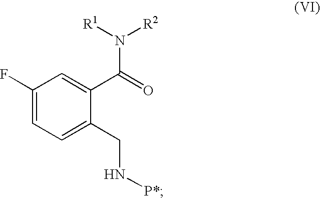
(Z) treating the benzamide compound of Formula (VI) with an amine deprotecting agent to obtain the benzamide compound of Formula (VII);
wherein:
R¹ and R² are each independently:
(1) —H,
(2) —C₁₋₆ alkyl, optionally substituted with from 1 to 5 substituents each of which is independently —OH, —O—C₁₋₆ alkyl, —CN, —NO₂, —N(R^{a})R^{b}, —C(═O)N(R^{a})R^{b}, —SO₂N(R^{a})R^{b}, —N(R^{a})C(═O)R^{b}, —N(R^{a})CO₂R^{c}, —N(R^{a})SO₂R^{c}, —N(R^{a})SO₂N(R^{a})R^{b}, —OC(═O)N(R^{a})R^{b}, or —N(R^{a})C(═O)N(R^{a})R^{b},
(3) —C₃₋₆ cycloalkyl, optionally substituted with from 1 to 4 substituents each of which is independently —C₁₋₄ alkyl or —O—C₁₋₄ alkyl, or
(4) aryl, optionally substituted with from 1 to 6 substituents each of which is independently halogen, —C₁₋₄ alkyl, —O—C₁₋₄ alkyl, —CN, —N(R^{a})R^{b}, —C(═O)N(R^{a})R^{b}, —SO₂N(R^{a})R^{b}, —N(R^{a})C(═O)R^{b}, —N(R^{a})CO₂R^{c}, —N(R^{a})SO₂R^{c}, —(CH₂)₁₋₂—O—C₁₋₄ alkyl, —(CH₂)₁₋₂—CN, —(CH₂)₁₋₂—N(R^{a})R^{b}, —(CH₂)₁₋₂—C(═O)N(R^{a})R^{b}, —(CH₂)₁₋₂—SO₂N(R^{a})R^{b}, —(CH₂)₁₋₂—N(R^{a})C(═O)R^{b}, —(CH₂)₁₋₂—N(R^{a})CO₂R^{c}, —(CH₂)₁₋₂—N(R^{a})SO₂R^{c}, phenyl, or —(CH₂)₁₋₂-phenyl;
R³ is —C₁₋₆ alkyl, —C₁₋₆ alkyl-aryl, or aryl;
P* is an amino protective group;
each R^{a} is independently —H, —C₁₋₆ alkyl, or —C₃₋₆ cycloalkyl;
each R^{b} is independently —H, —C₁₋₆ alkyl, or —C₃₋₆ cycloalkyl; and
each R^{c} is independently —C₁₋₆ alkyl or —C₃₋₆ cycloalkyl.

2. The process according to claim 1, wherein R¹ and R² are each independently —H, —C₁₋₆ alkyl, —C₃₋₆ cycloalkyl, or aryl.

3. The process according to claim 1, wherein P* is
(1) —C(═O)—O—C₁₋₆ alkyl,
(2) —C(═O)—O—CH₂-aryl,
(3) —C(═O)—O—(CH₂)₀₋₁—CH═CH₂, 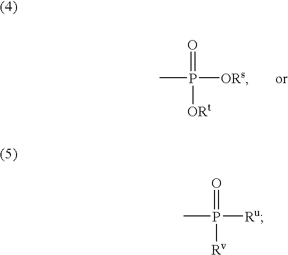
wherein R^{s} and R^{t} are each independently —C₁₋₆ alkyl, —CH₂-aryl, or aryl; and
R^{u} and R^{v} are each independently an aryl group.

4. The process according to claim 1, wherein the reaction in Step Y is conducted at a temperature in a range of from about 50 to about 200° C.

5. The process according to claim 1, wherein the amine of formula R¹R²NH is employed in Step Y in an amount in a range of from about 1 to about 200 equivalents per equivalent of benzoate compound V.

6. The process according to claim 1, wherein the solvent Y is selected from the group consisting of aromatic hydrocarbons, halogenated aliphatic hydrocarbons, alcohols, ethers, and nitrites.

7. The process according to claim 1, wherein P* is an amino protective group capable of being cleaved by an acid and the amine deprotecting agent in Step Z comprises an acid Z that is employed in an amount in a range of from about 0.1 to about 100 equivalents per equivalent of benzamide compound VI; and the treatment in Step Z is conducted at a temperature in a range of from about −50 to about 150° C.

8. The process according to claim 1, which further comprises:
(X) treating a benzoate compound of Formula (IV): 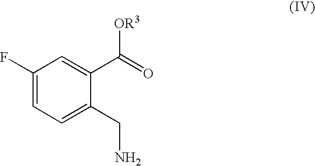
with an amine protecting agent containing the group P* in a solvent X to obtain the benzoate compound of Formula (V).

9. The process according to claim 8, which further comprises:
(W) hydrogenating a benzonitrile of Formula (III): 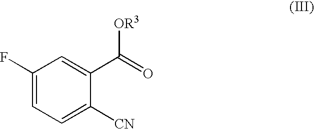
in a solvent W and in the presence of a transition metal catalyst to obtain the benzoate compound of Formula (IV).

10. The process according to claim 9, which further comprises:
(V) reacting a halobenzoate compound of Formula (II): 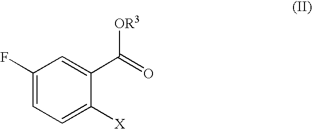
in an aprotic solvent V with a cyanide compound selected from the group consisting of CuCN and Zn(CN)₂ to obtain the benzonitrile of Formula (III); with the proviso that when the cyanide compound is Zn(CN)₂, the reaction is conducted in the presence of a Pd compound and an activating ligand; wherein X is chloro, bromo, or iodo.

11. The process according to claim 10, which further comprises:
(U) esterifying a benzoic acid of Formula (I): 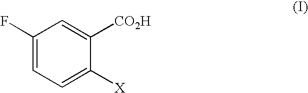
with an alcohol of formula R³—OH optionally in the presence of an acid U to obtain the halobenzoate compound of Formula (II).

12. The process according to claim 1, wherein P* is BOC, ALLOC, or CBZ; and wherein the process further comprises:
(XA) hydrogenating a benzonitrile of Formula (III): 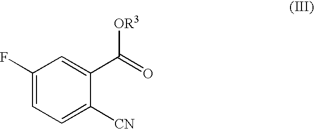
in a solvent XA, in the presence of (i) (BOC)₂O, (ALLOC)₂O, or (CBZ)₂O and (ii) Raney nickel, and optionally in the presence of a base to obtain a benzoate compound of Formula (V):

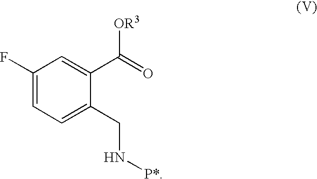

13. A process for preparing Compound 7: 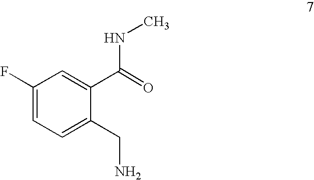
which comprises:
(yy) reacting a benzoate compound of Formula (Va): 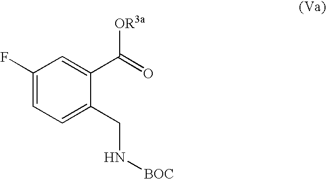
with methylamine in a solvent yy to obtain Compound 6: 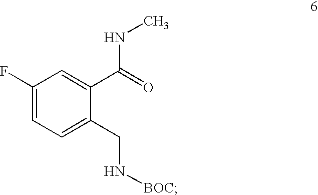
and
(zz) treating the Compound 6 with an acid zz to obtain the Compound 7;
wherein R^{3a} is —C₁₋₆ alkyl.

14. The process according to claim 13, wherein the benzoate compound of Formula (Va) is Compound 5:

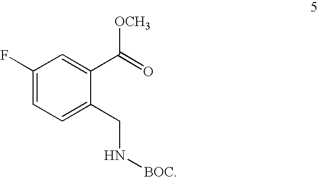

15. The process according to claim 13, wherein:
the reaction in Step yy is conducted at a temperature in the range of from about 75 to 150° C.;
methylamine is employed in Step yy in an amount in a range of from about 1.5 to about 5 equivalents per equivalent of Compound Va;
the solvent yy is selected from the group consisting of alcohols, ethers, and aromatic hydrocarbons
the acid zz is HCl;
the acid zz is employed in Step zz in an amount in a range of from about 3 to about 15 equivalents per equivalent of Compound 6; and
the treatment in Step zz is conducted in a solvent zz which is an C₁₋₆ alkyl ester of a C₁₋₆ alkylcarboxylic acid.

16. The process according to claim 13, which further comprises (xx) treating a benzoate compound of Formula (IVa): 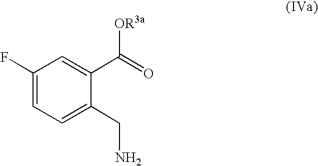
with an amine protecting agent containing the BOC group in a solvent xx to obtain the benzoate compound of Formula (Va).

17. The process according to claim 16, which further comprises (ww) hydrogenating a benzonitrile of Formula (IIIa): 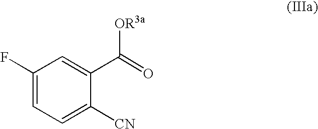
in a solvent ww and in the presence of a transition metal catalyst to obtain the benzoate compound of Formula (IVa).

18. The process according to claim 17, which further comprises:
(vv) reacting a halobenzoate compound of Formula (IIa): 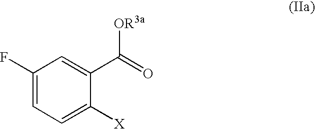
in an aprotic solvent vv with a cyanide compound selected from the group consisting of CuCN and Zn(CN)₂ to obtain the benzonitrile of Formula (IIIa); with the proviso that when the cyanide compound is Zn(CN)₂, the reaction is conducted in the presence of a Pd compound and an activating ligand; wherein X is chloro, bromo, or iodo.

19. The process according to claim 13, which further comprises:
(xxa) hydrogenating a benzonitrile of Formula (IIIa): 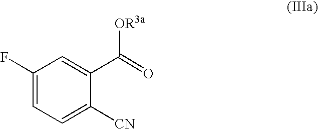
in a solvent xxa, in the presence of (BOC)₂O and Raney nickel, and optionally in the presence of a base to obtain a benzoate compound of Formula (Va):

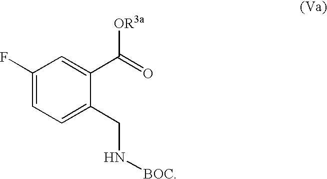

20. A compound selected from the group consisting of:
a compound of Formula (Va): 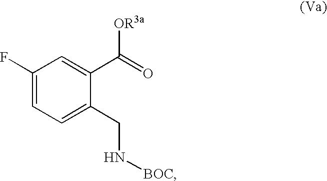
a compound of Formula (IVa): 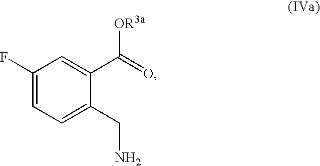
a salt of a compound of Formula (IVa), and
a compound of Formula (IIIa): 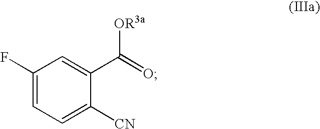
wherein R^{3a} is —C₁₋₆ alkyl.
